(19) United States
(12) Patent Application Publication
(10) Pub. No.: US 2023/0158136 A1
(43) Pub. Date: May 25, 2023

(54) VIRAL NUCLEIC ACID MOLECULES, AND COMPOSITIONS AND METHODS OF USE THEREOF

(71) Applicants: Versitech Limited, Hong Kong (CN); Centre for Virology, Vaccinology and Therapeutics Limited, Hong Kong (CN)

(72) Inventors: Hanjun Zhao, Hong Kong (CN); Kwok-Yung YUEN, Hong Kong (CN)

(21) Appl. No.: 18/058,169

(22) Filed: Nov. 22, 2022

Related U.S. Application Data
(60) Provisional application No. 63/283,101, filed on Nov. 24, 2021.

(51) Int. Cl. A61K 39/145; C12N 7/00

(52) U.S. Cl. CPC A61K 39/145; C12N 7/00; C12N 2760/16121; C12N 2760/16122; C12N 2760/16134; A61K 2039/5252

(57) ABSTRACT

The disclosure relates to defective interfering genes and viruses thereof. It has been discovered that defective interfering genes designed from influenza and coronavirus species can inhibit infection or replication of the parent virus. In vivo, DIGs induce rapid-onset prophylactic protection of infected animals against lethal viral doses. Thus, disclosed herein are nucleic acids containing DIGs, pharmaceutical compositions thereof and associated methods of use. For example, described herein is an isolated polynucleotide containing one or more defective interfering genes, wherein each of the one or more defective interfering genes contains a nucleotide sequence corresponding to one or more portions of an influenza or coronavirus gene or genome, wherein the nucleotide sequence includes a deletion in the gene. The DIGs can be in the form of a plasmid. Pharmaceutical compositions of the plasmid can be used to limit viral replication and prevent or treat influenza or coronavirus associated diseases.

### CROSS-REFERENCE TO RELATED APPLICATIONS

This application claims priority to and benefit of U.S. Provisional Application No. 63/283,101, filed Nov. 24, 2021. Application No. 63/283,101, filed Nov. 24, 2021, is hereby incorporated herein by reference in its entirety.

### REFERENCE TO SEQUENCE LISTING

The Sequence Listing submitted Nov. 22, 2022, as a text file named “UHK_01130_US_ST26.xml”, created Oct. 18, 2022, and having a size of 89,128 bytes is hereby incorporated by reference pursuant to 37 C.F.R § 1.834(c)(1).

### FIELD OF THE INVENTION

The disclosed invention generally relates to antiviral agents, and specifically, compositions and methods for prevention and/or treatment of viral infection and diseases.

### BACKGROUND OF THE INVENTION

Clinical studies demonstrated that early treatment or prophylactic treatment might be more effective methods for controlling these diseases. Apart from therapeutic antivirals being developed, prophylactic antivirals may play important roles in controlling infectious diseases. Vaccination is currently the most effective strategy in preventing viral infection, but vaccine effectiveness can be strain dependent and historically, has varied from 10-60% protection in the past decades for influenza [Centers for Disease Control and Prevention, Update: drug susceptibility of swine-origin influenza A (H1N1) viruses, MMWR Morb Mortal Wkly Rep, 58, 433-435 (2009)]. This may be similar for the COVID-19 vaccine given the continual circulation of SARS-CoV-2 variants. The onset of effective vaccine protection requires at least two weeks to generate sufficient titers of neutralizing antibodies. The neuraminidase inhibitors, zanamivir and oseltamivir, against influenza viruses are approved for prophylaxis, but both need to be administrated daily and there are concerns about drug resistance if these neuraminidase inhibitors are frequently used. Antiviral drugs directly targeting SARS-CoV-2 have shown benefits for patients with the mild or moderate symptoms but not for patients presenting with severe symptoms [Beigel, J. H. et al., The New England Journal of Medicine, 383, 1813-1826, Doi: 10.1056/NEJMoa2007764, (2020); Spinner, C. D. et al., JAMA, 324(11), 1048-1057, Doi: 10.1001/jama.2020.16349, (2020); Thiruchelvam, K. et al., Expert Rev Anti Infect Ther, 1-19, Doi: 10.1080/14787210.2021.1949984, (2021)]. This may indicate that early treatment or prophylactic treatment should be considered for COVID-19 cases.

Defective interfering genes (DIGs) are viral genes with internal deletions [Huang, A. S. & Baltimore, D. Nature, 226, 325-327, Doi: 10.1038/226325a0, (1970)]. Influenza DIG could inhibit the replication of the cognate full-length viral RNA and the antiviral activity is affected by the length and the origin of the DIG [Meng, B. et al., Virology journal 14, 138, Doi: 10.1186/s12985-017-0805-6 (2017); Duhaut, S. D. & Dimmock, N. J. J Gen Virol 83, 403-411, Doi: 10.1099/0022-1317-83-2-403, (2002); Dimmock, N. J., et al., J Virol 82 (17), 8570-8578, Doi: 10.1128/JVI.00743-08 (2008); Bdeir, N. et al. PLoS One, 14, e0212757, Doi: 10.1371/journal.pone.0212757, (2019); Zhao, H. et al. Nat Commun, 9, 2358, Doi: 10.1038/s41467-018-04792-7, (2018)]. The identification of sub-genomic RNAs of coronaviruses suggested that coronaviruses could generate defective genes during viral replication. However, it is not clear which DIG works best for anti-influenza activity and if there is any DIG which can significantly inhibit coronavirus in vitro and in vivo.

There is a need to develop prophylactic antivirals with new antiviral mechanisms and broad-spectrum antiviral effects for emerging influenza virus and coronavirus, especially for people with risk factors of susceptibility and/or severe diseases.

It is an object of the invention to provide antiviral compositions and methods of use thereof.

It is another object of the invention to provide compositions and methods for delivering nucleic acids encoding defective interfering genes.

It is another object of the invention to provide compositions and methods for prevention and/or treatment of viral diseases, including individual diseases caused by influenza viruses and coronaviruses, including SARS-CoV-2.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### BRIEF SUMMARY OF THE INVENTION

Potent antiviral agents to protect against pathogenic viral species, as well as their variants have been developed. The working examples demonstrate development of defective interfering genes (DIGs) designed from influenza and coronavirus species which can significantly inhibit infection and replication of the cognate viruses. When tested in vivo, DIGs from influenza or coronavirus induce rapid-onset prophylactic protection of infected animals, thus providing an alternative to the current clinical strategies for anti-influenza and coronavirus prophylaxis. Thus, disclosed herein are nucleic acids containing DIGs, pharmaceutical compositions thereof and associated methods of use.

In particular, disclosed is an isolated polynucleotide containing one or more defective interfering genes, wherein each of the one or more defective interfering genes contains a nucleotide sequence corresponding to one or more portions of a viral gene or genome, wherein the nucleotide sequence includes a deletion in the gene. More specifically, the portion of the viral gene comprises a deletion relative to the viral gene. Preferably, the virus is an influenza virus (e.g., influenza A, influenza B, or influenza C) or coronavirus (e.g., SARS-CoV1, SARS-CoV2, MERS-CoV and other coronaviruses).

In some forms, when the virus is an influenza virus, the gene encodes an RNA polymerase or subunit thereof, such as, but not limited to PA, PB1, and PB2. In some forms, the nucleotide sequence can include an internal (central) deletion in the gene. More specifically, the portion of the viral gene comprised in the nucleotide sequence comprises an internal deletion relative to the viral gene. In some forms, the nucleotide sequence includes about 150-600 nucleotides from the 5′ end of the gene, about 150-600 nucleotides from the 3′ end of the gene, or a combination thereof. Preferably, the nucleotide sequence includes about 450 nucleotides from the 5′ end of the gene and about 450 nucleotides from the 3′ end of the gene.

In particular forms, the one or more defective interfering genes includes the nucleotide sequence of any one of SEQ ID NOs:7-11, 14-16, or 18, or a nucleotide sequence having 75% or more sequence identity to any one of SEQ ID NOs: 7-11, 14-16, or 18. In some forms, the polynucleotide collectively includes the nucleotide sequence of SEQ ID NO:14, SEQ ID NO:15, and SEQ ID NO:16.

In some forms, when the virus is a coronavirus, for example, a β-coronavirus, more preferably SARS-CoV-2, the gene encodes a structural, non-structural, or accessory protein or fragment thereof selected from ORF1a, ORF1b, S, M, ORF3a, ORF6, ORF7a, ORFS, and ORF10. In some forms, the nucleotide sequence includes about 600-1200 nucleotides from the 5′ end of the coronavirus genome, about 600-1200 nucleotides from the 3′ end of the coronavirus genome, about 600-1200 nucleotides from the gene encoding ORF1b, or a combination thereof. In some forms, the one or more defective interfering genes includes the nucleotide sequence of SEQ ID NO:5 or SEQ ID NO:6, or a nucleotide sequence having 75% or more sequence identity to SEQ ID NO:5 or SEQ ID NO:6.

In any of the foregoing the deletion can include about 400-2000 nucleotides or about 3,000-27,000 nucleotides relative to the virus upon which the DIG is based. In some forms, the deletion contains contiguous nucleotides. In some forms, the deletion contains non-contiguous nucleotides.

Vectors are also provided. In some forms, the vector contains a disclosed polynucleotide. The vector can further include one or more promoters and/or polyadenylation signals operably linked to the one or more defective interfering genes contained in the polynucleotide. Preferably, the vector is an expression vector, such as a plasmid.

Compositions containing a disclosed polynucleotide or vector are also described. In some forms, the composition further includes a peptide. Suitable peptides include TAT-P1 (YGRKKRRQRRRCWGPCPTAFRQIGNCGRFRVRCCRIR; SEQ ID NO:3), TAT2-P1 (RKKRRQRRRCWGPCPTAFRQIGNCGRFRVRCCRIR; SEQ ID NO:4), LAH4: KKALLAHALHLLALLALHLAHALKKA-NH2 (SEQ ID NO:83), or a combination thereof. In some forms, the peptide to polynucleotide weight ratio in the composition is in the range of about 2:1 to 4:1. Preferably, the weight ratio is 4:1.

In some forms, the composition can also comprise one or more additional polynucleotides as disclosed or one or more additional vectors as disclosed. In some forms, the composition comprises three of the disclosed vectors, where the first vector comprises the nucleotide sequence of SEQ ID NO:12, where the second vector comprises the nucleotide sequence of SEQ ID NO:13, and where the third vector comprises the nucleotide sequence of SEQ ID NO:14.

In some forms, the peptide complexes with the polynucleotide to form a nanoparticle. Thus, the disclosed compositions can include peptide-polynucleotide nanoparticles. In some forms, the nanoparticles have an average diameter of less than 200 nm or less than 150 nm. In some forms, the nanoparticles have an average diameter of about 135 nm.

Pharmaceutical compositions are also provided. In some forms, the pharmaceutical compositions include any of the foregoing composition in combination with a pharmaceutically acceptable carrier or excipient.

Methods of using the disclosed nucleic acids and compositions thereof are described. Disclosed is a method of producing one or more defective interfering genes involving introducing a disclosed vector to a host cell and incubating the host cell under conditions sufficient for expression of the polynucleotide, thereby producing the one or more defective interfering genes.

Also provided is a method of reducing replication of an influenza or coronavirus in a cell involving introducing a disclosed vector to the cell under conditions suitable for the cell to produce defective viruses containing one or more RNAs transcribed from the polynucleotide, thereby reducing replication of the virus.

Disclosed is a method of treating an influenza or coronavirus infection in a subject by administering to the subject an effective amount of a disclosed pharmaceutical composition. Also described is a method of preventing or treating an influenza or coronavirus associated disease in a subject involving administering to the subject an effective amount of a pharmaceutical composition. In some forms, the subject has been exposed to, is infected with, or is at risk of infection by the influenza virus or coronavirus. In some forms, the subject is immunocompromised.

The influenza virus can be selected from influenza A or influenza B. Suitable influenza strains include H1N1, H2N2, H3N2, H3N8, H5N1 or H7N9. In some forms, the coronavirus is SARS-CoV-1 or SARS-CoV-2. Suitable SARS-CoV-2 strains or variants include SARS-CoV-2 HKU-001a, SARS-CoV-2 B.1.1.7 (Alpha variant), SARS-CoV-2 B.1.351 (Beta variant), SARS-CoV-2 B.1.617.1 (Kappa variant), SARS-CoV-2 B.1.617.2 (Delta variant), and SARS-CoV-2 B.1.617.3

In any of the foregoing methods, the composition can be administered via oral, intranasal or intratracheal administration. Preferably, wherein the subject is human

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A is a schematic illustrating the construction of defective interfering genes (DIGs). The DIGs of DI-PB2, DI-PB1 and DI-PA with internal deletion were generated by fusion PCR. Dotted lines indicate the internal deletion in wild-type viral polymerase genes. The indicated solid-line sequences of shortened viral polymerase gene PB2, PB1 and PA were inserted into the PHW2000 vector. Figure IB is a bar graph showing the antiviral activity of the various DIGs against A(H7N7) virus in A549 cells. FIG. 1C is a bar graph showing the dose-dependent antiviral efficiency of single PAD4 and combinatorial DIG-3 (including PADS, PB2D3, and PB1D3) and DI-PAD4 (including PAD4, PB2D3, and PB1D3). The plasmid DIGs or empty vector (PHW) with indicated concentrations were transfected into A549 cells and then cells were infected by H7N7 virus at 24 h post transfection. Viral titers in cell supernatants at 48 hours post infection were measured via a plaque assay. FIG. 1D is a survival curve showing the protection of two doses of DIGs on A(H1N1)-infected mice. The DI-PAD4, PAD4, DIG-3, or empty vector (PHW) packaged by TAT1-P1 were intratracheally inoculated into corresponding mice at 48 hours and 24 hours before viral inoculation. Survival curves were generated from 5 mice in each group. FIG. 1E is a survival curve showing the protection of one dose of DIGs on A(H1N1)-infected mice. The DI-PAD4, PAD4, DIG-3, or PHW packaged by TAT1-P1 were intratracheally inoculated into mice at 24 hours before viral inoculation. Survival curves were generated from 10 mice in each group.

FIG. 2A illustrates the construction of a coronavirus DIG. Coronavirus DIGs (CD2100 and CD3600) were synthesized according to the parent sequence of HKU-001a and were inserted into vector PHW2000. Dotted lines indicate the internal deletion in wild-type HKU001a (SARS-CoV-2). The three solid-line sequences linked together were inserted into the PHW2000 vector to generate CD2100 and CD3600. FIG. 2B is a bar graph showing DIG RNA expression in 293T, Calu-3 and HK-2 cells. RNA expression in cells were measured by RT-qPCR by the specific CD2100 and CD3600 primers after 24 hours plasmid transfection. FIG. 2C is a bar graph illustrating the antiviral activity of DIG in HK-2 cells. SARS-CoV-2 (B.1.1.63) was added to DIG-transfected cells for viral replication. Viral titers in cell supernatants were measured via plaque assay 48 hours post infection. FIG. 2D is a bar graph showing the dose-dependent antiviral activity of CD3600. CD3600 or PHW (0.5 μg, 0.25 μg, or 0.125 μg/well) were transfected into HK-2 cells one day before viral infection. Viral titers in cell supernatants were measured at 48 hours post infection. FIG. 2E is a bar graph showing the antiviral activity of CD3600 during multi-cycle viral replication after viral passages. The supernatant viruses collected from HK-2 cells (transfected with PHW or CD3600 and infected with SARS-CoV-2) were passaged in Vero-E6 cells and viral titers in supernatants were determined at 24 hours post infection. FIG. 2F is a bar graph showing the broad-spectrum antiviral activity of CD3600 against five SARS-CoV-2 variants.

FIG. 3A is a bar graph showing the transfection efficiency of TAT-P1, TAT2-P1, and in vivo jetPEI in mouse lungs. The pCMV-Luc was packaged by the indicated vectors with a peptide:DNA weight ratio of 4:1. The luciferase expression in mouse lungs was measured at 24 hours post transfection. Luciferase expression in mouse lungs was normalized to 1 mg protein and TAT-P1 as 1000. Mock indicates mice treated with 5% glucose without pCMV-Luc. FIG. 3B is a bar graph showing the hydrodynamic diameter of nanoparticles of pDI-PAD4 packaged by the indicated vectors. Sizes were measured by DynaPro Plate Reader. FIG. 3C is a bar graph showing the luciferase expression in mouse lungs transfected with nanoparticle TAT2-P1/pCMV-Luc (1 mg ml-1/0.25 mg ml-1) and TAT2-P1/pCMV-Luc (2 mg ml-1/0.5 mg ml-1), respectively. Mock indicates mouse lungs inoculated with TAT2-P1 without DNA.

FIG. 4A is a survival curve showing the protective efficacy of antivirals administrated to mouse lungs 3 days before virus challenge. TAT2-P1/DI-PAD4 (20 μg/5 μg), zanamivir (40 μg), or TAT2-P1/PHW (20 μg/5 μg) were intratracheally inoculated into corresponding mice 3 days before A(H1N1)pdm09 virus challenge. For vaccine treatment, mice were intratracheally inoculated with TAT2-P1/PHW and intramuscularly injected with 480 ng of vaccine (Vaccine-480) 3 days before A(H1N1)pdm09 virus challenge. FIG. 4B illustrates the body weight changes of infected mice corresponding to FIG. 4A. FIG. 4C is a survival curve showing the protective efficacy of antivirals administrated to mouse lungs 5 days before viral challenge. TAT2-P1/PHW, TAT2-P1/DI-PAD4, or vaccine-480 were intratracheally inoculated into mice 5 days before A(H1N1)pdm09 virus challenge. FIG. 4D illustrates the body weight changes of infected mice corresponding to FIG. 4C. Survival curves were generated from 5-10 mice in each group. FIGS. 4E and 4F are bar graphs showing the viral titers in hamster lungs at day 2 post infection. TAT2-P1/CD3600 (50₁1g/12.5₁1g) was intranasally inoculated to hamster lungs at day 1 (CD3600-D1) or day 3 (CD3600-D3) before SARS-CoV-2 (Delta) challenge. TAT2-P1/PHW was given to hamster lungs at day 1 before viral challenges. Lung tissues were collected at day 2 post infection for measuring viral loads by plaque assay and RT-qPCR.

FIGS. 5A-5G are bar graphs of results demonstrating that TAT2-P1&LAH4 enhanced the gene expression and inhibited SARS-CoV-2 variants in vivo. FIG. 5A shows the luciferase expression in 293T cells. The pCMV-Luc was packaged by the indicated vectors (TAT2-P1, LAH4, and TAT2-P1:LAH4=3:2, 4:1 or 9:1) and was transfected into 293T cells (n=4). FIG. 5B shows the luciferase expression in mouse lungs (n=4). The pCMV-Luc was packaged by the indicated vectors and was inoculated to mouse lungs. The expression of luciferase in mouse lungs was detected at 24 hours post transfection. FIG. 5C shows the peptidic nanoparticle sizes of plasmids packaged by the indicated vectors (n=4). * indicates P<0.05 and ** indicates P<0.01. FIG. 5D shows that TAT2-P1&LAH4 can deliver CD3600 to significantly inhibit Omicron (n=3) variant replication in hamster lungs. One dose of CD3600 was intratracheally inoculated to hamster lungs at 1-day before viral challenge. Viral loads in hamster lungs were measured at 2-day post infection. FIG. 5E shows that TAT2-P1 significantly inhibit SARS-CoV-2 infection (n=4) in VeroE6 cells. SARS-CoV-2 treated by indicated peptides for plaque reduction assay. PFU (%) was plaque number of peptide-treated virus normalized to plaque number of untreated virus. ** indicates P<0.01. FIG. 5F shows two doses of PBS (n=4), TAT2-P1 (n=3), TAT2-P1&LAH4 with CD3600 (n=4) or PHW (n=3) were given to hamster lungs at 1-day before and 8 hours after Omicron SARS-CoV-2 infection. FIG. 5G shows that PBS (n=4) or TAT2-P1&LAH4 with CD3600 (n=4) were given to hamster lungs at 1-day before and 8 hours after Delta SARS-CoV-2 infection. Viral loads were measured at 2-day post infection. * indicates P<0.05 and ** indicates P<0.01. P values were calculated by the two-tailed Student's t test.

FIG. 6 is a bar graph showing the cytotoxicity of TAT2-P1 and LAH4 in 293T cells. The indicated concentrations of peptides were incubated into 293T cells for 24 hours. Cell viability (%) was the OD value of peptide-treated cells normalized to that of cells without treatment. The cytotoxicity was measured by MTT assay. Data were presented as mean ±SD of four biological samples.

FIG. 7 is a bar graph of the nanoparticle stability for cell transfection. Luciferase plasmids packaged by TAT2-P1&LAH4 (4:1) were prepared for 72 hours, 24 hours and 15 minutes before the transfection in 293T cells. After transfection, the luciferase expression in cells were measured at 24 hours post transfection. Data were presented as mean ±SD of at least four biological samples.

### DETAILED DESCRIPTION OF THE INVENTION

The disclosed method and compositions may be understood more readily by reference to the following detailed description of particular embodiments and the Examples included therein and to the Figures and their previous and following description.

It has been discovered that that defective interfering genes of influenza virus delivered by TAT-P1 to mouse lungs could protect mice from influenza A virus infection [Zhao, H. et al. Nat Commun, 9, 2358, Doi: 10.1038/s41467-018-04792-7, (2018)]. The working Examples show that DIG antiviral activity in lungs was related to the antiviral activity of DIGs and the transfection efficiency of DIG in lung tissue. TAT mediated transfection is mainly through the endocytosis, including clathrin-mediated endocytosis [Richard, J. P. et al., J Biol Chem 280, 15300-15306, Doi: 10.1074/jbc.M401604200, (2005)], caveolae/lipid-raft-mediated endocytosis [Ferrari, A. et al., Mol Ther 8, 284-294; Doi: 10.1016/s1525-0016(03)00122-9 , (2003)], micropinocytosis [Wadia, J. S., et al., Nat Med 10, 310-315; Doi: 10.1038/nm996, (2004)], and endocytosis independent pathways [Duchardt, F. et al., Traffic 8, 848-866, Doi: 10.1111/j.1600-0854.2007.00572.x. (2007)]. In the airway and lungs, the passive cellular uptakes of nanoparticles are mainly mediated through endocytosis pathways and affected by the sizes of nanoparticles because of the barrier effect from airway mucus [Wang, Z. et al., ACS Nano 3, 4110-4116, Doi: 10.1021/nn9012274, (2009); Zhao, F. et al. Small 7, 1322-1337, Doi: 10.1002/smll.201100001, (2011); Duncan, G. A., et al., Mol Ther 24, 2043-2053, Doi: 10.1038/mt.2016.182 (2016); Foroozandeh, P. & Aziz, A. A. Nanoscale Res Lett 13, 339, Doi: 10.1186/s11671-018-2728-6 (2018)]. One feasible way to increase the uptake efficiency in airway and lung is to optimize the nanoparticle size to reduce the barrier effect of mucus so as to overcome the limit set by the average pore sizes (100-200 nm) in airway mucus [Duncan, G. A., et al., Mol Ther 24, 2043-2053, Doi: 10.1038/mt.2016.182 (2016); Mastorakos P. et al., Proc Natl Acad Sci USA 112, 8720-8725, Doi: 10.1073/pnas.1502281112 (2015)].

The study described in the working Examples demonstrate that a defective interfering influenza PA gene (PAD4) had the best antiviral activity in human A549 cells when compared with twelve other DIGs from polymerase genes of segmented-RNA influenza virus. Treatment of mice with the combination of three defective interfering genes, DIG-4 (including PAD4, PB1D3, and PB2D3) conferred the best protection against A(H1N1)pdm09 virus when compared with that of mice treated by PAD4 or DIG-3. It was also found that a TAT2-P1 vector could more efficiently transfect plasmid in vivo with less toxicity compared with the TAT-P1 vector. Consequently, a single dose of TAT2-P1/CD3600 could inhibit SARS-CoV-2 replication in hamsters when CD3600 was given to hamster lungs at 1 day prior to a viral challenge. It was also observed that a single dose of TAT2-P1/DIG-4 could efficiently protect 90% and 50% mice from a lethal influenza viral infection when DIG-4 was given to mice at 3-day and 5-day prior to a viral challenge respectively.

Results from these experiments demonstrate that a single dose of DIGs for coronavirus or influenza virus can provide rapid-onset prophylactic protection in infected animals, providing an alternative to the current clinical strategies for anti-coronavirus and influenza virus prophylaxis. Because of the low possibility of DIG inducing drug resistant viruses [Dimmock, N. J. & Easton, A. J. Journal of virology 88, 5217-5227, Doi: 10.1128/JVI.03193-13, (2014)], DIGs may circumvent the drug-resistance challenges associated with current approaches.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### I. Definitions

“Defective interfering” in the context of genes or genomes refers to genes that are derived from or associated with a parent virus. These genes are called “defective” because they have lost the capacity to code for all the viral proteins necessary for independent replication and thus are defective in the absence of the parent (also called helper) virus. Accordingly, the helper virus is required to provide the missing replication protein(s) in trans. DIGs are referred as “interfering” because they can attenuate the symptoms caused by the helper virus. Typically, DIGs harbor one or more deletions relative to the parent virus gene or genome. Thus, DIGs can have non-contiguous portions of their corresponding helper virus' genes or genomes.

The terms “SARS-CoV-2” and “Severe Acute Respiratory Syndrome Coronavirus 2” refer to the pathogenic coronavirus strains of the subgenus Sarbecovirus which are derived from the beta-coronavirus of zoonotic origin which emerged in Asia in late 2019, and which are the causative agents of pandemic Coronavirus disease 2019 (COVID-19) in humans. SARS-CoV-2 viruses have a high rate of genetic mutation within the genome, resulting in rapid development of multiple variant SARS-CoV-2 virus strains. Multiple variants of the virus that causes COVID-19 have been documented globally during this pandemic, including a variant called B.1.1.7 identified in the United Kingdom, a variant called B.1.351 identified in South Africa, and a variant called P.1 identified in Brazil.

The terms “influenza virus”, “influenza” and “flu virus” are used interchangeably and refer to the group of influenza virus A, influenza virus B, influenza virus C and influenza virus D. Human influenza A and B viruses cause seasonal epidemics of disease (termed the “flu season”) in humans almost every winter in the United States. Global epidemics of flu disease are termed “Flu pandemics”, and typically occur when a new and very different influenza A virus emerges that both infects humans and has the ability to spread efficiently between humans. Influenza A viruses are categorized as either the hemagglutinin subtype or the neuraminidase subtype based on the proteins involved. There are 18 distinct subtypes of hemagglutinin and 11 distinct subtypes of neuraminidase. Influenza A is the primary cause of flu epidemics.

“Corresponds” or “corresponding” in the context of nucleic acids refers to the relationship in which a first nucleotide sequence encodes the same product as a second nucleotide sequence. The two nucleotide sequences need not be identical in order to correspond, but they can be identical. One of the nucleotide sequences can be designed based on the sequence of the other nucleotide sequence.

“Introduce,” as used herein, refers to bringing into contact. By “contact” or “contacting” is meant to allow or promote a state of immediate proximity or association between at least two elements. For example, to introduce a composition (e.g., a disclosed polynucleotide or vector containing a defective interfering gene) to a cell is to provide contact between the cell and the composition. The term encompasses penetration of the contacted composition to the interior of the cell by any suitable means, e.g., via transfection, electroporation, transduction, gene gun, nanoparticle delivery, etc.

The term “operably linked” or “operationally linked” refers to functional linkage between a regulatory sequence and a heterologous nucleic acid sequence permitting them to function in their intended manner (e.g., resulting in expression of the latter). The term encompasses positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site.

“Isolated” means altered or removed from the natural state. An isolated nucleic acid can exist in substantially purified form, or can exist in a non-native environment such as, for example, a host cell. An “isolated” nucleic acid encompasses a nucleic acid segment or fragment which has been separated from sequences which flank it in a naturally occurring state, e.g., a DNA fragment which has been removed from the sequences which are normally adjacent to the fragment in a genome in which it naturally occurs. The term also applies to nucleic acids which have been substantially purified from other components which naturally accompany the nucleic acid (e.g., RNA or DNA or proteins, which naturally accompany it in the cell). The term therefore includes, for example, a mRNA, or recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. Isolated does not require absolute purity, and can include protein, peptide, nucleic acid, or virus molecules that are at least 50% isolated, such as at least 75%, 80%, 90%, 95%, 98%, 99%, or even 99.9% isolate.

The terms “inhibit” or “reduce” means to decrease e.g., in activity, expression or levels. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “preventing” as used herein refers to administering a compound prior to the onset of clinical symptoms of a disease or conditions so as to prevent a physical manifestation of aberrations associated with the disease or condition.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like. “Expression vector” refers to a vector containing a polynucleotide having expression control sequences operatively linked to a nucleotide sequence to be expressed. An expression vector contains sufficient cis-acting elements for expression; other elements for expression can be supplied by the host cell or in an in vitro expression system. Expression vectors include all those known in the art, such as cosmids, plasmids (e.g., naked or contained in liposomes), phagemids, BACs, YACs, and viral vectors (e.g., vectors derived from lentiviruses, retroviruses, adenoviruses, and adeno-associated viruses) that incorporate the recombinant polynucleotide.

The term “percent (%) sequence identity” describes the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The % sequence identity of a given nucleic acid or amino acid sequence C to, with, or against a given nucleic acid or amino acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

The term “effective amount” means a quantity sufficient to provide a desired pharmacologic and/or physiologic effect. The exact amount required can vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “subject” refers to any individual, organism or entity. Typical subjects include animals (e.g., mammals such as mice, rats, rabbits, goats, pigs, chimpanzees, or horses, non-human primates, and humans) and/or plants. The term does not denote a particular age or sex. Thus, adult and newborn subjects, as well as fetuses, whether male or female, are intended to be covered. The subject may be healthy or suffering from or susceptible to a disease, disorder, or condition.

Recitation of ranges of values are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other forms the values may range in value either above or below the stated value in a range of approximately +/−5%; in other forms the values may range in value either above or below the stated value in a range of approximately +/−2%; in other forms the values may range in value either above or below the stated value in a range of approximately +/−1%. The preceding ranges are intended to be made clear by context, and no further limitation is implied.

### II. Compositions

1. Nucleic Acids

Disclosed are nucleic acids containing defective interfering genes (DIGs) designed from influenza and coronavirus species which can significantly inhibit infection and replication of the cognate viruses.

In particular, disclosed is an isolated polynucleotide containing one or more defective interfering genes, wherein each of the one or more defective interfering genes contains a nucleotide sequence corresponding to one or more portions of a viral gene or genome, wherein the nucleotide sequence includes a deletion in the gene. More specifically, the portion of the viral gene comprises a deletion relative to the viral gene. Preferably, the virus is an influenza virus (e.g., influenza A, influenza B, or influenza C) or coronavirus.

In some forms, when the virus is an influenza virus, the gene encodes an RNA polymerase or subunit thereof, such as, but not limited to PA, PB1, and PB2. In some forms, the nucleotide sequence can include an internal (central) deletion in the gene. More specifically, the portion of the viral gene included in the nucleotide sequence includes a deletion relative to the source viral gene. In some forms, the nucleotide sequence includes about 150-600 nucleotides from the 5′ end of the gene, about 150-600 nucleotides from the 3′ end of the gene, or a combination thereof. Preferred are internal deletions that render the gene segment incapable of producing a functional protein but are not so large as to hinder packaging of the gene segments of the virus into viral particles. The 150 nucleotides from both the 5′ and 3′ end is necessary for viral replication and packaging and are therefore retained. Even more preferably, the nucleotide sequence includes about 450 nucleotides from the 5′ end of the gene and about 450 nucleotides from the 3′ end of the gene.

In particular forms, the one or more defective interfering genes includes the nucleotide sequence of any one of SEQ ID NOs: 7-11, 14-16, or 18, or a nucleotide sequence having 75% or more sequence identity to any one of SEQ ID NOs: 7-11, 14-16, or 18. In some forms, the polynucleotide collectively includes the nucleotide sequence of SEQ ID NO:14, SEQ ID NO:15, and SEQ ID NO:16.

In some forms, when the virus is a coronavirus, for example, a β-coronavirus, more preferably SARS-CoV-2, the gene encodes a structural, non-structural, or accessory protein or fragment thereof selected from ORF1a, ORF1b, S, and M. In some forms, the nucleotide sequence includes about 600-1200 nucleotides from the 5′ end of the coronavirus genome, about 600-1200 nucleotides from the 3′ end of the coronavirus genome, about 600-1200 nucleotides from the gene encoding ORF1b, or a combination thereof. In some forms, the one or more defective interfering genes includes the nucleotide sequence of SEQ ID NO:5 or SEQ ID NO:6, or a nucleotide sequence having 75% or more sequence identity to SEQ ID NO:5 or SEQ ID NO:6.

In any of the foregoing the deletion can include about 400-2000 nucleotides or about 3,000-27,000 nucleotides relative to the virus upon which the DIG is based. In some forms, the deletion contains contiguous nucleotides. In some forms, the deletion contains non-contiguous nucleotides.

Compositions containing a disclosed polynucleotide or vector are also described. In some forms, the composition further includes a peptide. Suitable peptides include TAT-P1 (YGRKKRRQRRRCWGPCPTAFRQIGNCGRFRVRCCRIR; SEQ ID NO:3), TAT2-P1 (RKKRRQRRRCWGPCPTAFRQIGNCGRFRVRCCRIR; SEQ ID NO:4),

LAH4 (KKALLAHALHLLALLALHLAHALKKA-NH2; SEQ ID NO:83), or a combination thereof. In some forms, the peptide to polynucleotide weight ratio in the composition is in the range of about 2:1 to 4:1. Preferably, the weight ratio is 4:1.

In some forms, the peptide complexes with the polynucleotide to form a nanoparticle. Thus, the disclosed compositions can include peptide-polynucleotide nanoparticles. In some forms, the nanoparticles have an average diameter of less than 200 nm or less than 150 nm. In some forms, the nanoparticles have an average diameter of about 135 nm.

The influenza virus can be selected from influenza A or influenza B. Suitable influenza strains include H1N1, H2N2, H3N2, H3N8, H5N1or H7N9. In some forms, the coronavirus is SARS-CoV-2. Suitable SARS-CoV-2 strains or variants include SARS-CoV-2 HKU-001a, SARS-CoV-2 B.1.1.7 (Alpha variant), SARS-CoV-2 B.1.351 (Beta variant), SARS-CoV-2 B.1.617.1 (Kappa variant), SARS-CoV-2 B.1.617.2 (Delta variant), and SARS-CoV-2 B.1.617.3.
a. Coronavirus DIGs

In some embodiments, the DIG is derived from one or more coronaviruses. The coronaviruses (order Nidovirales, family Coronaviridae, and genus Coronavirus) are a diverse group of large, enveloped, positive-stranded RNA viruses that cause respiratory and enteric diseases in humans and other animals.

Coronaviruses typically have narrow host specificity and can cause severe disease in many animals, and several viruses, including infectious bronchitis virus, feline infectious peritonitis virus, and transmissible gastroenteritis virus, are significant veterinary pathogens. Human coronaviruses (HCoVs) are found in both group 1 (HCoV-229E) and group 2 (HCoV-OC43) and are historically responsible for ˜30% of mild upper respiratory tract illnesses.

At ˜30,000 nucleotides, their genome is the largest found in any of the RNA viruses. There are three groups of coronaviruses; groups 1 and 2 contain mammalian viruses, while group 3 contains only avian viruses. Within each group, coronaviruses are classified into distinct species by host range, antigenic relationships, and genomic organization. The genomic organization is typical of coronaviruses, with the characteristic gene order (5′-replicase [rep], spike [S], envelope [E], membrane [M], nucleocapsid [N]-3′) and short untranslated regions at both termini The SARS-CoV rep gene, which includes approximately two-thirds of the genome, encodes two polyproteins (encoded by ORFla and ORF1b) that undergo co-translational proteolytic processing. There are four open reading frames (ORFs) downstream of rep that are predicted to encode the structural proteins, S, E, M, and N, which are common to all known coronaviruses.

In some embodiments, the DIG is derived from a severe acute respiratory syndrome coronavirus-2 (SARS-CoV-2) betacoronavirus of the subgenus Sarbecovirus. SARS-CoV-2 viruses share approximately 75% genome sequence identity with the SARS-CoV virus identified in 2003. An exemplary nucleic acid sequence for the SARS-CoV-2 ORF1a/b gene is set forth in GenBank accession number MN908947.3. The genome organization of SARS-CoV-2 viruses is shared with other betacoronaviruses; six functional open reading frames (ORFs) are arranged in order from 5′ to 3′: replicase (ORF1a/ORF1b), spike (S), envelope (E), membrane (M) and nucleocapsid (N). In addition, seven putative ORFs encoding accessory proteins are interspersed between the structural genes.

In some preferred embodiments, the DIG includes one or more SARS-CoV-2 nucleic acid sequences from one or more of the genes encoding structural (S, E, M, N), or non-structural (NSPs) SARS-CoV-2 proteins. In some embodiments, the DIG includes one or more SARS-CoV-2 genes or gene expression products with selected epitopes in the SARS-CoV-2 genome that are conserved amongst multiple different coronaviruses. In some embodiments the SARS-CoV-2 variant is selected from SARS-CoV-2 B.1.1.7 (Alpha variant), SARS-CoV-2 B.1.351 (Beta variant), SARS-CoV-2 B.1.617, SARS-CoV-2 B.1.617.1 (Kappa variant), SARS-CoV-2 B.1.617.2 (Delta variant), and SARS-CoV-2 B.1.617.3.
b. Influenza Virus DIGs

In some embodiments, the DIG is a defective influenza virus particle. Influenza Virus DIGs can be derived from a particular influenza Glade or strain, or can be synthetic DIGs, designed to correspond with highly conserved genes amongst multiple different influenza virus strains.

There are four types of influenza viruses: A, B, C and D. Human influenza A and B viruses cause seasonal epidemics of disease. Influenza A viruses are the only influenza viruses known to cause flu pandemics, i.e., global epidemics of flu disease. Influenza type C infections generally cause mild illness and are not thought to cause human flu epidemics Influenza D viruses primarily affect cattle and are not known to infect or cause illness in people (see w.w.w.cdc.gov/flu/about/viruses/types.htm).

The influenza A virion is studded with glycoprotein spikes of hemagglutinin (HA) and neuraminidase (NA), in a ratio of approximately four to one, projecting from a host cell—derived lipid membrane. A smaller number of matrix (M2) ion channels traverse the lipid envelope, with an M2:HA ratio on the order of one M2 channel per 101-102 HA molecules. The envelope and its three integral membrane proteins HA, NA, and M2 overlay a matrix of M1 protein, which encloses the virion core. Internal to the M1 matrix are found the nuclear export protein (NEP; also called nonstructural protein 2, NS2) and the ribonucleoprotein (RNP) complex, which includes of the viral RNA segments coated with nucleoprotein (NP) and the heterotrimeric RNA-dependent RNA polymerase, composed of two “polymerase basic” and one “polymerase acidic” subunits (PB1, PB2, and PA). The organization of the influenza B virion is similar, with four envelope proteins: HA, NA, and, instead of M2, NB and BM2. Therefore, in some embodiments, the DIG is derived from one or more of the HA, NA, M2, NS2, NB, PB1, PB2, PA or NP genes of any influenza A or B virus. In particular embodiments, the DIG is derived from the PA gene of an influenza A or B virus (Bouvier and Palese P, Vaccine. 2008;26 Suppl 4(Suppl 4):D49-D53. doi:10.1016/j.vaccine.2008.07.039).

The influenza A and B virus genomes each include eight negative-sense, single-stranded viral RNA (vRNA) segments, while influenza C virus has a seven-segment genome. The eight segments of influenza A and B viruses (and the seven segments of influenza C virus) are numbered in order of decreasing length. In influenza A and B viruses, segments 1, 3, 4, and 5 encode just one protein per segment: the PB2, PA, hemagglutinin (HA) and nucleoprotein (NP) proteins. All influenza viruses encode the polymerase subunit PB1 on segment 2; in some strains of influenza A virus, this segment also codes for the accessory protein PB1-F2, a small, 87-amino acid protein with pro-apoptotic activity, in a +1 alternate reading frame. No analogue to PB1-F2 has been identified in influenza B or C viruses. Conversely, segment 6 of the influenza A virus encodes only the NA protein, while that of influenza B virus encodes both the NA protein and, in a −1 alternate reading frame, the NB matrix protein, which is an integral membrane protein corresponding to the influenza A virus M2 protein. Segment 7 of both influenza A and B viruses code for the M1 matrix protein. In the influenza A genome, the M2 ion channel is also expressed from segment 7 by RNA splicing, while influenza B virus encodes its BM2 membrane protein in a +2 alternate reading frame. Finally, both influenza A and B viruses possess a single RNA segment, segment 8, from which they express the interferon-antagonist NS1 protein and, by mRNA splicing, the NEP/NS2, which is involved in viral RNP export from the host cell nucleus. Influenza A viruses are divided into subtypes based on hemagglutinin (H) and neuraminidase (N) proteins on the surface of the virus. There are 18 different hemagglutinin subtypes and 11 different neuraminidase subtypes (H1 through H18, and N1 through N11, respectively). Therefore, in some embodiments, the DIG is derived from the PA gene of an influenza virus. In other embodiments, the DIG is derived from the PB1 and/or PB2 genes of an influenza. While there are potentially 198 different influenza A subtype combinations, only 131 subtypes have been detected in nature. Current subtypes of influenza A viruses that routinely circulate in people include A(H1N1) and A(H3N2). Therefore, in some embodiments, the DIG is derived from, or provides immunity to an A(H1N1) influenza virus, or an A(H3N2) influenza virus. In some embodiments, the DIG is conserved amongst, and/or provides immunity to all A(H1N1) influenza viruses. In some embodiments, the DIG is conserved amongst, and/or provides immunity to all A(H3N2) influenza viruses. In preferred embodiments, the antigen is conserved amongst, and/or provides immunity to A(H1N1) influenza viruses.

Influenza A viruses are further classified into multiple subtypes (e.g., H1N1, or H3N2), while influenza B viruses are classified into one of two lineages: B/Yamagata and B/Victoria. Both influenza A and B viruses can be further classified into specific clades and sub-clades. Clades and sub-clades can be alternatively called “groups” and “sub-groups,” respectively. An influenza Glade or group is a further subdivision of influenza viruses (beyond subtypes or lineages) based on the similarity of their HA gene sequences. Clades and subclades are shown on phylogenetic trees as groups of viruses that usually have similar genetic changes (i.e., nucleotide or amino acid changes) and have a single common ancestor represented as a node in the tree. Clades and sub-clades that are genetically different from others are not necessarily antigenically different (i.e., viruses from a specific Glade or sub-Glade may not have changes that impact host immunity in comparison to other clades or sub-clades). In some embodiments, the DIG is conserved amongst, and/or provides immunity to two or more influenza viruses within the same subtype and/or sub-Glade. In preferred embodiments, the DIG is conserved amongst, and/or provides immunity to two or more influenza viruses within different subtypes and/or sub-clades. In some embodiments, the DIG is conserved amongst, and/or provides immunity to all influenza viruses within the same subtype and/or sub-Glade. In preferred embodiments, the DIG is conserved amongst, and/or provides immunity to multiple influenza viruses within different subtypes and/or sub-clades.

Currently circulating influenza, A(H1N1) viruses are related to the pandemic 2009 H1N1 virus that emerged in spring of 2009 and caused a flu pandemic (See w.w.w.cdc.gov/flu/about/viruses/types.htm). This virus is known as “A(H1N1)pdm09 virus,” or “2009 H1N1,” and continued to circulate seasonally from 2009 to 2021. These H1N1 viruses have undergone relatively small genetic changes and changes to their antigenic properties over time.

Of the influenza viruses that circulate and cause human disease, influenza A(H3N2) viruses tend to change more rapidly, both genetically and antigenically and have formed many separate, genetically different clades that continue to co-circulate. Therefore, in some embodiments, the DIG is derived from and/or provides immunity to all currently circulating H1N1 influenza viruses. In some embodiments, the DIG is derived from and/or provides immunity to all currently circulating H3N2 influenza viruses. In preferred embodiments, the DIG is derived from and/or provides immunity to all currently circulating H1N1 influenza viruses and H3N2 influenza viruses. In some embodiments, the DIG is derived from an Influenza A virus PA gene, or an Influenza A virus PA gene expression product.

Influenza B viruses are classified into two lineages: B/Yamagata and B/Victoria. Influenza B viruses are further classified into specific clades and sub-clades. Influenza B viruses change more slowly in terms of genetic and antigenic properties than influenza A viruses. Surveillance data from recent years shows co-circulation of influenza B viruses from both lineages in the United States and around the world with. Therefore, in some embodiments, the DIG is derived from and/or provides immunity to influenza B viruses. In some embodiments, the DIG is derived from and/or provides immunity to all currently circulating influenza B viruses. In some embodiments, the DIG is derived from an Influenza B virus NP gene, or an Influenza B virus PA gene expression product.

In some embodiments, the DIG is derived from and/or provides immunity to B/Yamagata and B/Victoria influenza viruses. In other embodiments, the DIG is derived from and/or provides immunity to one or more H1N1 influenza virus, and to one or more influenza B virus. In other embodiments, the DIG is derived from and/or provides immunity to one or more H3N2 influenza virus, and to one or more influenza B virus. In other embodiments, the DIG is derived from and/or provides mucosal immunity to one or more H1N1 influenza virus, to one or more H3N2 influenza virus, and to one or more influenza B virus.
c. Other Viral DIGs

In some embodiments, the DIG is isolated from a virus including, but not limited to, a virus from any of the following viral families Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae, Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae.

Viral DIGs can be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

2. Expression Vectors

In some embodiments, the polynucleotide is incorporated into or part of a vector. Methods to construct expression vectors containing genetic sequences and appropriate transcriptional and translational control elements are well known in the art. These methods include in vitro recombinant DNA techniques, synthetic techniques, and in vivo genetic recombination. Expression vectors generally contain regulatory sequences and necessary elements for the translation and/or transcription of the inserted coding sequence, which can be, for example, the polynucleotide of interest. The coding sequence can be operably linked to a promoter and/or enhancer to help control the expression of the desired gene product. Promoters used in biotechnology are of different types according to the intended type of control of gene expression. They can be generally divided into constitutive promoters, tissue-specific or development-stage-specific promoters, inducible promoters, and synthetic promoters.

For example, in some embodiments, the nucleic acid encoding the defective interfering particle is operably linked to a promoter or other regulatory elements known in the art. Thus, the polynucleotide can be a vector such as an expression vector. The engineering of polynucleotides for expression in a prokaryotic or eukaryotic system may be performed by techniques generally known to those of skill in recombinant expression. An expression vector typically includes one of the compositions under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, mammalian cells and bacteria transformed with recombinant phage DNA, plasmid DNA or cosmid DNA expression vectors. It will be appreciated that any of these vectors may be used to package and deliver the DIGs.

Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HindIII site toward the BgII site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the compositions. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

In preferred embodiments, the polynucleotide cargo is an RNA, such as an mRNA. The mRNA can encode a polypeptide of interest In some embodiments, the mRNA has a cap on the 5′ end and/or a 3′ poly(A) tail which can modulate ribosome binding, initiation of translation and stability mRNA in the cell.

3. Pharmaceutical Formulations

Also provided are pharmaceutical formulations including any of the foregoing composition in combination with a pharmaceutically acceptable carrier or excipient.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, desintegrators, fillers, and coating compositions. For detailed information concerning materials, equipment, and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, Pa.: Williams & Wilkins, 1995).

### III. Methods of Making

Methods of making the disclosed nucleic acids compositions containing the defective interfering genes and pharmaceutical formulations thereof are described herein. Examples of methods of making and verifying the disclosed nucleic acid DIGs are described in the Examples. Typically, the defective interfering virus RNA for incorporation into the virus is produced by recombinant means. In some forms, the vector contains a disclosed polynucleotide. The vector can further include one or more promoters and/or polyadenylation signals operably linked to the one or more defective interfering genes contained in the polynucleotide. Preferably, the vector is an expression vector, such as a plasmid. Many techniques and methods useful for making the disclosed DIGs, polynucleotides, vectors, and compositions are known and came be adapted for such use.

Disclosed is a method of producing one or more defective interfering genes involving introducing a disclosed vector to a host cell and incubating the host cell under conditions sufficient for expression of the polynucleotide, thereby producing the one or more defective interfering genes.

### IV. Methods of Use

Methods of using the disclosed nucleic acids compositions containing the defective interfering genes and pharmaceutical formulations thereof are also provided. Provided is a method of reducing replication of an influenza or coronavirus in a cell involving introducing a disclosed vector to the cell under conditions suitable for the cell to produce defective viruses containing one or more RNAs transcribed from the polynucleotide, thereby reducing replication of the virus.

In some forms, the method can be used to treat an influenza or coronavirus infection in a subject by administering to the subject an effective amount of a disclosed pharmaceutical composition. In some forms, the method can be used to prevent or treat an influenza or coronavirus associated disease in a subject involving administering to the subject an effective amount of a pharmaceutical composition. In some forms, the subject has been exposed to, is infected with, or is at risk of infection by the influenza virus or coronavirus. In some forms, the subject is immunocompromised.

In some forms, the method includes a defective interfering gene for use as an antiviral agent. The influenza virus can be selected from influenza A or influenza B. Suitable influenza strains include H1N1, H2N2, H3N2, H3N8, H5N1 or H7N9. In some forms, the coronavirus is SARS-CoV1 and SARS-CoV-2. Suitable SARS-CoV-2 strains or variants include SARS-CoV-2 HKU-001a, SARS-CoV-2 B.1.1.7 (Alpha variant), SARS-CoV-2 B.1.351 (Beta variant), SARS-CoV-2 B.1.617.1 (Kappa variant), SARS-CoV-2 B.1.617.2 (Delta variant), and SARS-CoV-2 B.1.617.3. In all cases, the sequences are presented in the positive (antigenome sense) from 5′ to 3′. The sequences are also represented as DNA.

The disclosed defective interfering viral genes can also be used to induce host immune response. For example, the disclosed defective interfering viral genes can be used in vaccine compositions. The disclosed defective interfering viral genes can also be used for broad-spectrum antiviral activity by inducing host immune responses.

In any of the foregoing methods, the composition can be administered via oral, intranasal or intratracheal administration. Preferably, wherein the subject is human.

### V. Kits

The disclosed polynucleotides, reagents, compositions, and other materials can be packaged together in any suitable combination as a kit useful for performing, or aiding in the performance of, the methods. It is useful if the components in a given kit are designed and adapted for use together in the method.

For example, kits including vaccines or other compositions for administration to a subject, may include a pre-measured dosage of the composition in a sterile needle, ampule, tube, container, or other suitable vessel. The kits may include instructions for dosages and dosing regimens. In some forms, the compositions are lyophilized The kit may further include agents (e.g., saline, a buffered solution) and instructions to form a formulation for administration. The instructions may specify suitable storage conditions for the kit and components thereof.

Also provided are kits for protein production. Such kits can include a disclosed polynucleotide (e.g., plasmid or other expression vector), viruses, and/or instructions for use. The kit can further include reagents and instructions for transfection or transduction of recipient cells.

The disclosed compositions and methods can be further understood through the following numbered paragraphs.

1. An isolated polynucleotide comprising one or more defective interfering genes, wherein each of the one or more defective interfering genes comprises a nucleotide sequence corresponding to one or more portions of a viral gene, wherein the portion of the viral gene comprises a deletion relative to the viral gene, wherein the virus is an influenza virus or coronavirus.

2. The polynucleotide of paragraph 1, wherein the virus is an influenza A virus, influenza B virus, or influenza C virus.

3. The polynucleotide of paragraph 2, wherein the gene encodes an RNA polymerase or subunit thereof selected from PA, PB1, and PB2.

4. The polynucleotide of any one of paragraphs 1-3, wherein the portion of the viral gene comprises an internal deletion relative to the viral gene.

5. The polynucleotide of any one of paragraphs 1-4, wherein the nucleotide sequence comprises about 150-600 nucleotides from the 5′ end of the gene, about 150-600 nucleotides from the 3′ end of the gene, or a combination thereof.

6. The polynucleotide of paragraph 5, wherein the nucleotide sequence comprises about 450 nucleotides from the 5′ end of the gene and about 450 nucleotides from the 3′ end of the gene.

7. The polynucleotide of any one of paragraphs 1-6, wherein at least one of the one or more defective interfering genes comprises the nucleotide sequence of any one of SEQ ID NOs:5-10, 13-15, or 17, or a nucleotide sequence having 75% or more sequence identity to any one of SEQ ID NOs: 5-10, 13-15, or 17.

8. The polynucleotide of any one of paragraphs 1-7, wherein the polynucleotide collectively comprises the nucleotide sequence of SEQ ID NO:12, SEQ ID NO:13, and SEQ ID NO:14.

9. The polynucleotide of paragraph 1, wherein the virus is a coronavirus, preferably a β-coronavirus, more preferably SARS-CoV-2.

10. The polynucleotide of paragraph 9, wherein the gene encodes a structural, non-structural, accessory protein, or a fragment thereof selected from ORF1a, ORF1b, S, M, ORF3a, ORF6, ORF7a, ORFS, and ORF10.

11. The polynucleotide of paragraph 9 or 10, wherein the nucleotide sequence comprises about 600-1200 nucleotides from the 5′ end of the coronavirus genome, about 600-1200 nucleotides from the 3′ end of the coronavirus genome, about 600-1200 nucleotides from the gene encoding ORF1b, or a combination thereof.

12. The polynucleotide of any one of paragraphs 9-11, wherein at least one of the one or more defective interfering genes comprises the nucleotide sequence of SEQ ID NO:5 or SEQ ID NO:6, or a nucleotide sequence having 75% or more sequence identity to SEQ ID NO:5 or SEQ ID NO:6.

13. The polynucleotide of any one of paragraphs 1-12, wherein the deletion comprises about 400-2000 nucleotides or about 3,000-27,000 nucleotides, optionally wherein the nucleotides are contiguous or non-contiguous.

polynucleotide

14. A vector comprising the polynucleotide of any one of paragraphs 1-13, wherein the vector comprises one or more promoters and/or polyadenylation signals operably linked to the one or more defective interfering genes.

15. The vector of paragraph 14, wherein the vector is an expression vector, preferably a plasmid.

16. A method of producing one or more defective interfering genes, the method comprising introducing the vector of paragraph 14 or 15 to a host cell and incubating the host cell under conditions sufficient for expression of the polynucleotide, thereby producing the one or more defective interfering genes.

17. A method of reducing replication of an influenza virus or coronavirus in a cell, the method comprising introducing the vector of paragraph 14 or 15 to the cell under conditions suitable for the cell to produce defective viruses comprising one or more RNAs transcribed from the polynucleotide, thereby reducing replication of the virus.

18. A composition comprising the polynucleotide of any one of paragraphs 1-4, or the vector of paragraph 14 or 15.

19. The composition of paragraph 18 further comprising a peptide selected from TAT-P1 (YGRKKRRQRRRCWGPCPTAFRQIGNCGRFRVRCCRIR; SEQ ID NO:3), TAT2-P1 (RKKRRQRRRCWGPCPTAFRQIGNCGRFRVRCCRIR; SEQ ID NO:4), LAH4 (KKALLAHALHLLALLALHLAHALKKA-NH2; SEQ ID NO:83), or a combination thereof.

20. The composition of paragraph 19, wherein the peptide:polynucleotide weight ratio is in the range of about 2:1 to 4:1, preferably, 4:1.

21. The composition of paragraph 19 or 20, wherein the peptide complexes with the polynucleotide to form a plurality of nanoparticles.

22. The composition of paragraph 21, wherein the nanoparticles have an average diameter of less than 200 nm, preferably less than 150 nm, more preferably about 135 nm.

23. The composition of any one of paragraphs 18-22 further comprising one or more additional polynucleotides of paragraphs 1-4 or one or more additional vectors of paragraph 14 or 15.

24. The composition of paragraph 23, wherein the composition comprises three of the vectors, wherein the first vector comprises the nucleotide sequence of SEQ ID NO:12, wherein the second vector comprises the nucleotide sequence of SEQ ID NO:13, and wherein the third vector comprises the nucleotide sequence of SEQ ID NO:14.

25. A pharmaceutical composition comprising the composition of any one of paragraphs 17-24 and a pharmaceutically acceptable carrier or excipient.

26. A method of treating an influenza virus or coronavirus infection in a subject comprising administering to the subject an effective amount of the pharmaceutical composition of paragraph 25.

27. A method of preventing or treating an influenza virus or coronavirus associated disease in a subject comprising administering to the subject an effective amount of the pharmaceutical composition of paragraph 25.

28. The method of paragraph 26 or 27, wherein the subject has been exposed to, is infected with, or is at risk of infection by the influenza virus or coronavirus.

29. The method of any one of paragraphs 26-28, wherein the subject is immunocompromised.

30. The method of any one of paragraphs 26-29, wherein the influenza virus is influenza A virus or influenza B virus, or wherein the coronavirus is SARS-CoV-2.

31. The method of paragraph 30, wherein the influenza A virus is selected from H1N1, H2N2, H3N2, H3N8, H5N1, and H7N9.

32. The method of paragraph 30, wherein the SARS-CoV-2 is selected from SARS-CoV-2 HKU-001a, SARS-CoV-2 B.1.1.7 (Alpha variant), SARS-CoV-2 B.1.351 (Beta variant), SARS-CoV-2 B.1.617.1 (Kappa variant), SARS-CoV-2 B.1.617.2 (Delta variant), and SARS-CoV-2 B.1.617.3.

33. The method of any one of paragraphs 26-32, wherein the composition is administered via oral, intranasal, or intratracheal administration.

34. The method of any one of paragraphs 26-33, wherein the subject is human.

### EXAMPLES

### Example 1: Generation and Characterization of Antiviral DIGs Which Show Potent Activity Against Influenza and SARS-CoV-2 Viruses

### Materials and Methods

Cell and Virus Cultures

Madin Darby canine kidney (MDCK, ATCC CCL-34), 293T (ATCC CRL-3216), A549 (ATCC CCL-185), Vero-E6 (ATCC CRL-1586), VeroE6-TMPRSS2 (VeroE6-T), Calu-3 (ATCC HTB-55)[Zhao et al., Nat Commun 12, 1517, doi: 10.1038/s41467-021-21825-w (2021)] and HK-2 cells [Yeung et al., Cell, 184, 2212-2228.e2212, Doi: 10.1016/j.cell.2021.02.053 (2021)] were cultured in Dulbecco minimal essential medium (DMEM) or DMEM-F12K supplemented with 10% fetal bovine serum (FBS), 100 IU ml⁻¹ penicillin and 100 μg ml⁻¹ streptomycin. The virus strains used in this study included A/Hong Kong/415742Md/2009 (H1N1) [Zhao, H. et al., Sci Rep 6, 22008, Doi: 10.1038/srep22008 (2016)], A/Netherlands/219/2003 (H7N7) [Zhao, H. et al., Virology 498, 1-8, 10.1016/j.virol.2016.08.004 (2016)], and SARS-CoV-2 variants [Zhao, H. et al., Nat Commun 11, 4252, Doi: 10.1038/s41467-020-17986-9 (2020) and Chen, L. L. et al., Clin Infect Dis, Doi: 10.1093/cid/ciab656 (2021)]. Influenza viruses were cultured in MDCK cells, while SARS-CoV-2 were cultured in VeroE6 or VeroE6-T cells and viral titers and viral titers were determined by plaque assay.

Coronavirus DIG Construction and Expression

Based on previous studies which identified the possible packaging genes for coronavirus [Qin, L. et al., Acta Pharmacol Sin, 24, 489-496 (2003); Masters, P. S. Virology 537, 198-207, Doi: 10.1016/j.virol.2019.08.031 (2019); Seim, I., Roden, C. A. & Gladfelter, A. S. bioRxiv, Doi: 10.1016/j.bpj.2021.06.018 (2021)], the 5′ -end sequences (˜700 and 1200 bp), the internal sequences in ORF 1b and the 3′ -end sequences of SARS-CoV-2 of HKU-001a were synthesized (GenBank: MT230904.1, see Table 1). The synthesized genes were inserted into the PHW2000 vector to generate plasmid CD2100 and CD3600 for expressing defective genes of SARS-CoV-2. The DNA sequences of the constructed plasmids with CoV-DIG were verified by Sanger sequencing. CD2100 and CD3600 expression in 293T, A549 and HK-2 cells were measured by RT-qPCR after the plasmids were transfected into cells at 24 hours post transfection. For determining DIG RNA expression, all RNA samples were treated with DNase I (QIAGEN, Cat^{#} 79254) according to the manufacturer's instructions and further purified using a RNeasy Mini Kit (Qiagen, Cat^{#} 74106). The RNA expression levels were detected by RT-qPCR with DIG primers (see Table 2).

TABLE 1
Sequences of CoV DIGs.

| Gene | Oligonucleotide sequence (5′ to 3′) |
|---|---|
| CD2100 | ATTAAAGGTTTATACCTTCCCAGGT |
| | AACAAACCAACCAACTTTCGATCTC |
| | TTGTAGATCTGTTCTCTAAACGAAC |
| | TTTAAAATCTGTGTGGCTGTCACTC |
| | GGCTGCATGCTTAGTGCACTCACGC |
| | AGTATAATTAATAACTAATTACTGT |
| | CGTTGACAGGACACGAGTAACTCGT |
| | CTATCTTCTGCAGGCTGCTTACGGT |
| | TTCGTCCGTGTTGCAGCCGATCATC |
| | AGCACATCTAGGTTTCGTCCGGGTG |
| | TGACCGAAAGGTAAGATGGAGAGCC |
| | TTGTCCCTGGTTTCAACGAGAAAAC |
| | ACACGTCCAACTCAGTTTGCCTGTT |
| | TTACAGGTTCGCGACGTGCTCGTAC |
| | GTGGCTTTGGAGACTCCGTGGAGGA |
| | GGTCTTATCAGAGGCACGTCAACAT |
| | CTTAAAGATGGCACTTGTGGCTTAG |
| | TAGAAGTTGAAAAAGGCGTTTTGCC |
| | TCAACTTGAACAGCCCTATGTGTTC |
| | ATCAAACGTTCGGATGCTCGAACTG |
| | CACCTCATGGTCATGTTATGGTTGA |
| | GCTGGTAGCAGAACTCGAAGGCATT |
| | CAGTACGGTCGTAGTGGTGAGACAC |
| | TTGGTGTCCTTGTCCCTCATGTGGG |
| | CGAAATACCAGTGGCTTACCGCAAG |
| | GTTCTTCTTCGTAAGAACGGTAATA |
| | AAGGAGCTGGTGGCCATAGTTACGG |
| | CGCCGATCTAAAGTCATAACAGGGT |
| | GAAGTACCAGTTTCTATCATTAATA |
| | ACACTGTTTACACAAAAGTTGATGG |
| | TGTTGATGTAGAATTGTTTGAAAAT |
| | AAAACAACATTACCTGTTAATGTAG |
| | CATTTGAGCTTTGGGCTAAGCGCAA |
| | CATTAAACCAGTACCAGAGGTGAAA |
| | ATACTCAATAATTTGGGTGTGGACA |
| | TTGCTGCTAATACTGTGATCTGGGA |
| | CTACAAAAGAGATGCTCCAGCACAT |
| | ATATCTACTATTGGTGTTTGTTCTA |
| | TGACTGACATAGCCAAGAAACCAAC |
| | TGAAACGATTTGTGCACCACTCACT |
| | GTCTTTTTTGATGGTAGAGTTGATG |
| | GTCAAGTAGACTTATTTAGAAATGC |
| | CCGTAATGGTGTTCTTATTACAGAA |
| | GGTAGTGTTAAAGGTTTACAACCAT |
| | CTGTAGGTCCCAAACAAGCTAGTCT |
| | TAATGGAGTCACATTAATTGGAGAA |
| | GCCGTAAAAACACAGTTCAATTATT |
| | ATAAGAAAGTTGATGGTGTTGTCCA |
| | ACAATTACCTGAAACTTACTTTACT |
| | CAGAGTAGAAATTTACAAGAATTTA |
| | AACCCAGGAGTCAAATGGAAATTGA |
| | TTTCTTAGAATTAGCTATGGATGAA |
| | TTCATTGAACGGTATAAATTAGAAG |
| | GCTATGCCTTCGAACATATCGTTTA |
| | TGGAGATTTTAGTCATAGTCAGTTA |
| | GGTGGTTTACATCTACTGATTGGAC |
| | TAGCTAAACGTTTTAAGGAATCCGT |
| | TCTTCGGAATGTCGCGCATTGGCAT |
| | GGAAGTCACACCTTCGGGAACGTGG |
| | TTGACCTACACAGGTGCCATCAAAT |
| | TGGATGACAAAGATCCAAATTTCAA |
| | AGATCAAGTCATTTTGCTGAATAAG |
| | CATATTGACGCATACAAAACATTCC |
| | CACCAACAGAGCCTAAAAAGGACAA |
| | AAAGAAGAAGGCTGATGAAACTCAA |
| | GCCTTACCGCAGAGACAGAAGAAAC |
| | AGCAAACTGTGACTCTTCTTCCTGC |
| | TGCAGATTTGGATGATTTCTCCAAA |
| | CAATTGCAACAATCCATGAGCAGTG |
| | CTGACTCAACTCAGGCCTAAACTCA |
| | TGCAGACCACACAAGGCAGATGGGC |
| | TATATAAACGTTTTCGCTTTTCCGT |
| | TTACGATATATAGTCTACTCTTGTG |
| | CAGAATGAATTCTCGTAACTACATA |
| | GCACAAGTAGATGTAGTTAACTTTA |
| | ATCTCACATAGCAATCTTTAATCAG |
| | TGTGTAACATTAGGGAGGACTTGAA |
| | AGAGCCACCACATTTTCACCGAGGC |
| | CACGCGGAGTACGATCGAGTGTACA |
| | GTGAACAATGCTAGGGAGAGCTGCC |
| | TATATGGAAGAGCCCTAATGTGTAA |
| | AATTAATTTTAGTAGTGCTATCCCC |
| | ATGTGATTTTAATAGCTTCTTAGGA |
| | GAATGACAAAAAAAAAAAAAAAAAA |
| | AA (SEQ ID NO: 5) |
| CD3600 | ATTAAAGGTTTATACCTTCCCAGGT |
| | AACAAACCAACCAACTTTCGATCTC |
| | TTGTAGATCTGTTCTCTAAACGAAC |
| | TTTAAAATCTGTGTGGCTGTCACTC |
| | GGCTGCATGCTTAGTGCACTCACGC |
| | AGTATAATTAATAACTAATTACTGT |
| | CGTTGACAGGACACGAGTAACTCGT |
| | CTATCTTCTGCAGGCTGCTTACGGT |
| | TTCGTCCGTGTTGCAGCCGATCATC |
| | AGCACATCTAGGTTTCGTCCGGGTG |
| | TGACCGAAAGGTAAGATGGAGAGCC |
| | TTGTCCCTGGTTTCAACGAGAAAAC |
| | ACACGTCCAACTCAGTTTGCCTGTT |
| | TTACAGGTTCGCGACGTGCTCGTAC |
| | GTGGCTTTGGAGACTCCGTGGAGGA |
| | GGTCTTATCAGAGGCACGTCAACAT |
| | CTTAAAGATGGCACTTGTGGCTTAG |
| | TAGAAGTTGAAAAAGGCGTTTTGCC |
| | TCAACTTGAACAGCCCTATGTGTTC |
| | ATCAAACGTTCGGATGCTCGAACTG |
| | CACCTCATGGTCATGTTATGGTTGA |
| | GCTGGTAGCAGAACTCGAAGGCATT |
| | CAGTACGGTCGTAGTGGTGAGACAC |
| | TTGGTGTCCTTGTCCCTCATGTGGG |
| | CGAAATACCAGTGGCTTACCGCAAG |
| | GTTCTTCTTCGTAAGAACGGTAATA |
| | AAGGAGCTGGTGGCCATAGTTACGG |
| | CGCCGATCTAAAGTCATTTGACTTA |
| | GGCGACGAGCTTGGCACTGATCCTT |
| | ATGAAGATTTTCAAGAAAACTGGAA |
| | CACTAAACATAGCAGTGGTGTTACC |
| | CGTGAACTCATGCGTGAGCTTAACG |
| | GAGGGGCATACACTCGCTATGTCGA |
| | TAACAACTTCTGTGGCCCTGATGGC |
| | TACCCTCTTGAGTGCATTAAAGACC |
| | TTCTAGCACGTGCTGGTAAAGCTTC |
| | ATGCACTTTGTCCGAACAACTGGAC |
| | TTTATTGACACTAAGAGGGGTGTAT |
| | ACTGCTGCCGTGAACATGAGCATGA |
| | AATTGCTTGGTACACGGAACGTTCT |
| | GAAAAGAGCTATGAATTGCAGACAC |
| | CTTTTGAAATTAAATTGGCAAAGAA |
| | ATTTGACACCTTCAATGGGGAATGT |
| | CCAAATTTTGTATTTCCCTTAAATT |
| | CCATAATCAAGACTATTCAACCAAG |
| | GGTTGAAAAGAAAAAGCTTGATGGC |
| | TTTATGGGTAGAATTCGATCTGTCT |
| | ATCCAGTTGCGTCACCAAATGAATG |
| | AAAGTCTGCTACGTGTATAACACGT |
| | TGCAATTTAGGTGGTGCTGTCTGTA |
| | GACATCATGCTAATGAGTACAGATT |
| | GTATCTCGATGCTTATAACATGATG |
| | ATCTCAGCTGGCTTTAGCTTGTGGG |
| | TTTACAAACAATTTGATACTTATAA |
| | CCTCTGGAACACTTTTACAAGACTT |
| | CAGAGTTTAGAAAATGTGGCTTTTA |
| | ATGTTGTAAATAAGGGACACTTTGA |
| | TGGACAACAGGGTGAAGTACCAGTT |
| | TCTATCATTAATAACACTGTTTACA |
| | CAAAAGTTGATGGTGTTGATGTAGA |
| | ATTGTTTGAAAATAAAACAACATTA |
| | CCTGTTAATGTAGCATTTGAGCTTT |
| | GGGCTAAGCGCAACATTAAACCAGT |
| | ACCAGAGGTGAAAATACTCAATAAT |
| | TTGGGTGTGGACATTGCTGCTAATA |
| | CTGTGATCTGGGACTACAAAAGAGA |
| | TGCTCCAGCACATATATCTACTATT |
| | GGTGTTTGTTCTATGACTGACATAG |
| | CCAAGAAACCAACTGAAACGATTTG |
| | TGCACCACTCACTGTCTTTTTTGAT |
| | GGTAGAGTTGATGGTCAAGTAGACT |
| | TATTTAGAAATGCCCGTAATGGTGT |
| | TCTTATTACAGAAGGTAGTGTTAAA |
| | GGTTTACAACCATCTGTAGGTCCCA |
| | AACAAGCTAGTCTTAATGGAGTCAC |
| | ATTAATTGGAGAAGCCGTAAAAACA |
| | CAGTTCAATTATTATAAGAAAGTTG |
| | ATGGTGTTGTCCAACAATTACCTGA |
| | AACTTACTTTACTCAGAGTAGAAAT |
| | TTACAAGAATTTAAACCCAGGAGTC |
| | AAATGGAAATTGATTTCTTAGAATT |
| | AGCTATGGATGAATTCATTGAACGG |
| | TATAAATTAGAAGGCTATGCCTTCG |
| | AACATATCGTTTATGGAGATTTTAG |
| | TCATAGTCAGTTAGGTGGTTTACAT |
| | CTACTGATTGGACTAGCTAAACGTT |
| | TTAAGGAATCACCTTTTGAATTAGA |
| | AGATTTTATTCCTATGGACAGTACA |
| | GTTAAAAACTATTTCATAACAGATG |
| | CGCAAACAGGTTCATCTAAGTGTGT |
| | GTGTTCTGTTATTGATTTATTACTT |
| | GATGATTTTGTTGAAATAATAAAAT |
| | CCCAAGATTTATCTGTAGTTTCTAA |
| | GGTTGTCAAAGTGACTATTGACTAT |
| | ACAGAAATTTCATTTATGCTTTGGT |
| | GTAAAGATGGCCATGTAGAAACATT |
| | ATACACCAAAAGATCACATTGGCAC |
| | CCGCAATCCTGCTAACAATGCTGCA |
| | ATCGTGCTACAACTTCCTCAAGGAA |
| | CAACATTGCCAAAAGGCTTCTACGC |
| | AGAAGGGAGCAGAGGCGGCAGTCAA |
| | GCCTCTTCTCGTTCCTCATCACGTA |
| | GTCGCAACAGTTCAAGAAATTCAAC |
| | TCCAGGCAGCAGTAGGGGAACTTCT |
| | CCTGCTAGAATGGCTGGCAATGGCG |
| | GTGATGCTGCTCTTGCTTTGCTGCT |
| | GCTTGACAGATTGAACCAGCTTGAG |
| | AGCAAAATGTCTGGTAAAGGCCAAC |
| | AACAACAAGGCCAAACTGTCACTAA |
| | GAAATCTGCTGCTGAGGCTTCTAAG |
| | AAGCCTCGGCAAAAACGTACTGCCA |
| | CTAAAGCATACAATGTAACACAAGC |
| | TTTCGGCAGACGTGGTCCAGAACAA |
| | ACCCAAGGAAATTTTGGGGACCAGG |
| | AACTAATCAGACAAGGAACTGATTA |
| | CAAACATTGGCCGCAAATTGCACAA |
| | TTTGCCCCCAGCGCTTCAGCGTTCT |
| | TCGGAATGTCGCGCATTGGCATGGA |
| | AGTCACACCTTCGGGAACGTGGTTG |
| | ACCTACACAGGTGCCATCAAATTGG |
| | ATGACAAAGATCCAAATTTCAAAGA |
| | TCAAGTCATTTTGCTGAATAAGCAT |
| | ATTGACGCATACAAAACATTCCCAC |
| | CAACAGAGCCTAAAAAGGACAAAAA |
| | GAAGAAGGCTGATGAAACTCAAGCC |
| | TTACCGCAGAGACAGAAGAAACAGC |
| | AAACTGTGACTCTTCTTCCTGCTGC |
| | AGATTTGGATGATTTCTCCAAACAA |
| | TTGCAACAATCCATGAGCAGTGCTG |
| | ACTCAACTCAGGCCTAAACTCATGC |
| | AGACCACACAAGGCAGATGGGCTAT |
| | ATAAACGTTTTCGCTTTTCCGTTTA |
| | CGATATATAGTCTACTCTTGTGCAG |
| | AATGAATTCTCGTAACTACATAGCA |
| | CAAGTAGATGTAGTTAACTTTAATC |
| | TCACATAGCAATCTTTAATCAGTGT |
| | GTAACATTAGGGAGGACTTGAAAGA |
| | GCCACCACATTTTCACCGAGGCCAC |
| | GCGGAGTACGATCGAGTGTACAGTG |
| | AACAATGCTAGGGAGAGCTGCCTAT |
| | ATGGAAGAGCCCTAATGTGTAAAAT |
| | TAATTTTAGTAGTGCTATCCCCATG |
| | TGATTTTAATAGCTTCTTAGGAGAA |
| | TGACAAAAAAAAAAAAAAAAAAAAA |
| | (SEQ ID NO: 6) |

TABLE 2
Primers for RT-qPCR.

| | | Oligonucleotide |
|---|---|---|
| Gene | Primer | sequence (5’ to 3’) |
| PAD4 | DI-PA-F | ATCTGAGAAGACACACATCCAC |
| | | SEQ ID NO: 20) |
| | DI-PA-R | GGACTCTCCAACGGGCCATGTT |
| | | (SEQ ID NO: 21) |
| PB1D3 | DI-PB1-F | CACCGAAACTGGAGCACCGCAAC |
| | | (SEQ ID NO: 22) |
| | DI-PB1-R | TTTGTTCATCTTCAAGTATTCCT |
| | | (SEQ ID NO: 23) |
| PB2D3 | DI-PB2-F | GATAACGGAAATGATTCCT |
| | | (SEQ ID NO: 24) |
| | DI-PB2-R | TCAGAACTGCGGACTCAAC |
| | | (SEQ ID NO: 25) |
| CD2100 | CD2100-F | CTTCGTAAGAACGGTAATAAAGG |
| | | (SEQ ID NO: 26) |
| | CD2100-R | CAAACAATTCTACATCAACACC |
| | | (SEQ ID NO: 27) |
| CD3600 | CD3600-F | ATAATCAAGACTATTCAACCAAGGG |
| | | (SEQ ID NO: 28) |
| | CD3600-R | TCATTAGCATGATGTCTACAGAC |
| | | (SEQ ID NO: 29) |
| SARS-CoV-2 | S-F | CCTACTAAATTAAATGATCTCTGC |
| | | TTTACT (SEQ ID NO: 30) |
| | S-R | CAAGCTATAACGCAGCCTGTA |
| | | (SEQ ID NO: 31) |

Influenza DIG Construction

DIGs were constructed with ˜150 nucleotides and 450 nucleotides located at the 3′ and 5′ ends based on previous studies of DIGs having ˜300 nucleotides located at the 3′ and 5′ ends [Zhao, H. et al. Dual-functional peptide with defective interfering genes effectively protects mice against avian and seasonal influenza. Nat Commun 9, 2358 (2018), see Table 3]. DIGs with about 225 nucleotides and 600 nucleotides at the 3′ and 5′ ends were further constructed based on the antiviral activities in A549 cells. The full-length sequence of wild-type A/WSN/1933 PA, PB1, and PB2 genes were used as the template to generate defective interfering PA, PB1, and PB2 genes (DI-PA, DI-PB1 and DI-PB2). Short gene segments at the 3′ and 5′ ends of DIGs were amplified by gene-specific primers designed by Primer Premier 5.0 (see Table 3). The amplified short gene fragments at the 3′ and 5′ ends were fused by fusion PCR [Heckman, K. L. & Pease, L. R. Gene splicing and mutagenesis by PCR-driven overlap extension. Nat Protoc 2, 924-932 (2007)] to generate DI-PA, DI-PB1, and DI-PB2 genes using two pairs of primers for each gene (see Table 4). The fused DI-PA, DI-PB1, and DI-PB2 genes (Table 3) were inserted into BsmBI/BsaI sites of PHW2000 vector to generate plasmids of DI-PA, DI-PB1, and DI-PB2, respectively. The DNA sequences of the constructed plasmids with DIGs were verified by Sanger sequencing.

TABLE 3

| Sequences of Flu DIGs. | |
|---|---|
| Gene | Oligonucleotide sequence (5′ to 3′) |
| PAD1 | AGCGAAAGCAGGTCAATTATATTCA |
| | ATATGGAAAGAATAAAAGAACTAAG |
| | GAATCTAATGTCGCAGTCTCGCACT |
| | CGCGAGATACTCACAAAAACCACCG |
| | TGGACCATATGGCCATAATCAAGAA |
| | GTACACATCAGGAAGACAGGAGAAG |
| | AAGGAGAGAAGGCTAATGTGCTAAT |
| | TGGGAAGGAGACGTGGTGTTGGTAA |
| | TGAAACGGAAACGGAACTCTAGCAT |
| | ACTTACTGACAGCCAGACAGCGACC |
| | AAAAGAATTCGGATGGCCATCAATT |
| | AGTGTCGAATAGTTTAAAAACGACC |
| | TTGTTTCTACT |
| | (SEQ ID NO: 7) |
| PB1D1 | AGCGAAAGCAGGCAAACCATTTGAA |
| | TGGATGTCAATCCGACTTTACTTTT |
| | CTTAAAAGTGCCAGCACAAAATGCT |
| | ATAAGCACAACTTTCCCTTATACTG |
| | GAGACCCTCCTTACAGCCATGGGAC |
| | AGGAACAGGATACACCATGGATACT |
| | GTCATTCCAGAGCCCGAATTGATGC |
| | ACGAATTGATTTCGAATCTGGAAGG |
| | ATAAAGAAAGAGGAGTTCACTGAGA |
| | TCATGAAGATCTGTTCCACCATTGA |
| | AGAGCTCAGACGGCAAAAATAGTGA |
| | ATTTAGCTTGTCCTTCATGAAAAAA |
| | TGCCTTGTTTCTACT |
| | (SEQ ID NO: 8) |
| PB2D1 | AGCGAAAGCAGGTACTGATTCAAAA |
| | TGGAAGATTTTGTGCGACAATGCTT |
| | CAATCCGATGATTGTCGAGCTTGCG |
| | GAAAAGGCAATGAAAGAGTATGGAG |
| | AGGACCTGAAAATCGAAACAAACAA |
| | ATTTGCAGCAATATGCACTCACTTG |
| | GAAGTGGCTATATGAAGCAATTGAG |
| | GAGTGCCTGATTAATGATCCCTGGG |
| | TTTTGCTTAATGCTTCTTGGTTCAA |
| | CTCCTTCCTCACACATGCATTGAGA |
| | TAGTTGTGGCAATGCTACTATTTGC |
| | TATCCATACTGTCCAAAAAAGTACC |
| | TTGTTTCTACT |
| | (SEQ ID NO: 9) |
| PAD2 | AGCGAAAGCAGGTACTGATTCAAAA |
| | TGGAAGATTTTGTGCGACAATGCTT |
| | CAATCCGATGATTGTCGAGCTTGCG |
| | GAAAAGGCAATGAAAGAGTATGGAG |
| | AGGACCTGAAAATCGAAACAAACAA |
| | ATTTGCAGCAATATGCACTCACTTG |
| | GAAGTGTGCTTCATGTATTCAGATT |
| | TTCACTTCATCGATGAGCAAGGCGA |
| | GTCAATAGTCGTAGAACTTGGCGAT |
| | CAAGAAAACTGCTTCTTATCGTTCA |
| | GGCTCTTAGGGACAACCTGGAACCT |
| | GGGACCTTTGATCTTGGGGGGCTAT |
| | ATGAAGCAATTGAGGAGTGCCTGAT |
| | TAATGATCCCTGGGTTTTGCTTAAT |
| | GCTTCTTGGTTCAACTCCTTCCTCA |
| | CACATGCATTGAGATAGTTGTGGCA |
| | ATGCTACTATTTGCTATCCATACTG |
| | TCCAAAAAAGTACCTTGTTTCTACT |
| | (SEQ ID NO: 10) |
| PB1D2 | AGCGAAAGCAGGCAAACCATTTGAA |
| | TGGATGTCAATCCGACTTTACTTTT |
| | CTTAAAAGTGCCAGCACAAAATGCT |
| | ATAAGCACAACTTTCCCTTATACTG |
| | GAGACCCTCCTTACAGCCATGGGAC |
| | AGGAACAGGATACACCATGGATACT |
| | GTCAACAGGACACATCAGTACTCAG |
| | AAAGGGGAAGATGGACAACAAACAC |
| | CGAAACTGGAGCACCGCAACTCAAC |
| | AATTCTTCCCCAGCAGTTCATACAG |
| | AAGACCAGTCGGGATATCCAGTATG |
| | GTGGAGGCTATGGTTTCCAGAGCCC |
| | GAATTGATGCACGAATTGATTTCGA |
| | ATCTGGAAGGATAAAGAAAGAGGAG |
| | TTCACTGAGATCATGAAGATCTGTT |
| | CCACCATTGAAGAGCTCAGACGGCA |
| | AAAATAGTGAATTTAGCTTGTCCTT |
| | CATGAAAAAATGCCTTGTTTCTACT |
| | (SEQ ID NO: 11) |
| PB2D2 | AGCGAAAGCAGGTCAATTATATTCA |
| | ATATGGAAAGAATAAAAGAACTAAG |
| | GAATCTAATGTCGCAGTCTCGCACT |
| | CGCGAGATACTCACAAAAACCACCG |
| | TGGACCATATGGCCATAATCAAGAA |
| | GTACACATCAGGAAGACAGGAGAAG |
| | AACCCAGCACTTAGGATGAAATGGA |
| | TGATGGCAATGAAATATCCAATTAC |
| | AGCAGACAAGAGGATAACGGAAATG |
| | GGGCAAAGAAGACAGGAGATATGGA |
| | CCAGCATTAAGCATAAATGAACTGA |
| | GCAACCTTGCGAAAGGAGAGAAGGC |
| | TAATGTGCTAATTGGGCAAGGAGAC |
| | GTGGTGTTGGTAATGAAACGGAAAC |
| | GGAACTCTAGCATACTTACTGACAG |
| | CCAGACAGCGACCAAAAGAATTCGG |
| | ATGGCCATCAATTAGTGTCGAATAG |
| | TTTAAAAACGACCTTGTTTCTACT |
| | (SEQ ID NO: 12) |
| PAD3 | AGCAAAAGCAGGTACTGATTCAAAA |
| | TGGAAGATTTTGTGCGACAATGCTT |
| | CAATCCGATGATTGTCGAGCTTGCG |
| | GAAAAGGCAATGAAAGAGTATGGAG |
| | AGGACCTGAAAATCGAAACAAACAA |
| | ATTTGCAGCAATATGCACTCACTTG |
| | GAAGTGTGCTTCATGTATTCAGATT |
| | TTCACTTCATCGATGAGCAAGGCGA |
| | GTCAATAGTCGTAGAACTTGGCGAT |
| | CCAAATGCACTTTTGAAGCACAGAT |
| | TTGAAATAATCGAGGGAAGAGATCG |
| | CACAATAGCCTGGACAGTAATAAAC |
| | AGTATTTGCAACACTACAGGGGCTG |
| | AGAAACCAAAGTTTCTACCAGATTT |
| | GTATGAGAGAGTCCCCCAAAGGAGT |
| | GGAGGAAGGTTCCATTGGGAAGGTC |
| | TGCAGAACTTTATTGGCAAAGTCGG |
| | TATTCAACAGCTTGTATGCATCTCC |
| | ACAACTGGAAGGATTTTCAGCTGAA |
| | TCAAGAAAACTGCTTCTTATCGTTC |
| | AGGCTCTTAGGGACAACCTGGAACC |
| | TGGGACCTTTGATCTTGGGGGGCTA |
| | TATGAAGCAATTGAGGAGTGCCTGA |
| | TTAATGATCCCTGGGTTTTGCTTAA |
| | TGCTTCTTGGTTCAACTCCTTCCTC |
| | ACACATGCATTGAGATAGTTGTGGC |
| | AATGCTACTATTTGCTATCCATACT |
| | GTCCAAAAAAGTACCTTGTTTCTAC |
| | T (SEQ ID NO: 13) |
| PB1D3 | AGCAAAAGCAGGCAAACCATTTGAA |
| | TGGATGTCAATCCGACTTTACTTTT |
| | CTTAAAAGTGCCAGCACAAAATGCT |
| | ATAAGCACAACTTTCCCTTATACTG |
| | GAGACCCTCCTTACAGCCATGGGAC |
| | AGGAACAGGATACACCATGGATACT |
| | GTCAACAGGACACATCAGTACTCAG |
| | AAAGGGGAAGATGGACAACAAACAC |
| | CGAAACTGGAGCACCGCAACTCAAC |
| | CCGATTGATGGGCCACTGCCAGAAG |
| | ACAATGAACCAAGTGGTTATGCCCA |
| | AACAGATCAAAAGAAATCGATCCAT |
| | CTTGAATACAAGCCAAAGAGGAATA |
| | CTTGAAGATGAACAAATGTACCAAA |
| | AGTGCTGCAACTTATTTGAAAAATT |
| | CTTCCCCAGCAGTTCATACAGAAGA |
| | CCAGTCGGGATATCCAGTATGGTGG |
| | AGGCTATGGTTTCCAGAGCCCGAAT |
| | TGATGCACGAATTGATTTCGAATCT |
| | GGAAGGATAAAGAAAGAGGAGTTCA |
| | CTGAGATCATGAAGATCTGTTCCAC |
| | CATTGAAGAGCTCAGACGGCAAAAA |
| | TAGTGAATTTAGCTTGTCCTTCATG |
| | AAAAAATGCCTTGTTTCTACT |
| | (SEQ ID NO: 14) |
| PB2D3 | AGCGAAAGCAGGTCAATTATATTCA |
| | ATATGGAAAGAATAAAAGAACTAAG |
| | GAATCTAATGTCGCAGTCTCGCACT |
| | CGCGAGATACTCACAAAAACCACCG |
| | TGGACCATATGGCCATAATCAAGAA |
| | GTACACATCAGGAAGACAGGAGAAG |
| | AACCCAGCACTTAGGATGAAATGGA |
| | TGATGGCAATGAAATATCCAATTAC |
| | AGCAGACAAGAGGATAACGGAAATG |
| | ATTCCTGAGAGAAATGAGCAGGGAC |
| | AAACTTTATGGAGTAAAATGAATGA |
| | CGCCGGATCAGACCGAGTGATGGTA |
| | TCACCTAACTGAAGACCCAGATGAA |
| | GGCACAGCTGGAGTTGAGTCCGCAG |
| | TTCTGAGAGGATTCCTCATTCTGGG |
| | CAAAGAAGACAGGAGATATGGACCA |
| | GCATTAAGCATAAATGAACTGAGCA |
| | ACCTTGCGAAAGGAGAGAAGGCTAA |
| | TGTGCTAATTGGGCAAGGAGACGTG |
| | GTGTTGGTAATGAAACGGAAACGGA |
| | ACTCTAGCATACTTACTGACAGCCA |
| | GACAGCGACCAAAAGAATTCGGATG |
| | GCCATCAATTAGTGTCGAATAGTTT |
| | AAAAACGACCTTGTTTCTACT |
| | (SEQ ID NO: 15) |
| PAD4 | AGCGAAAGCAGGTACTGATTCAAAA |
| | TGGAAGATTTTGTGCGACAATGCTT |
| | CAATCCGATGATTGTCGAGCTTGCG |
| | GAAAAGGCAATGAAAGAGTATGGAG |
| | AGGACCTGAAAATCGAAACAAACAA |
| | ATTTGCAGCAATATGCACTCACTTG |
| | GAAGTGTGCTTCATGTATTCAGATT |
| | TTCACTTCATCGATGAGCAAGGCGA |
| | GTCAATAGTCGTAGAACTTGGCGAT |
| | CCAAATGCACTTTTGAAGCACAGAT |
| | TTGAAATAATCGAGGGAAGAGATCG |
| | CACAATAGCCTGGACAGTAATAAAC |
| | AGTATTTGCAACACTACAGGGGCTG |
| | AGAAACCAAAGTTTCTACCAGATTT |
| | GTATGATTACAAGAAGAATAGATTC |
| | ATCGAAATTGGAGTAACAAGGAGAG |
| | AAGTTCACATATACTATCTGGAAAA |
| | GGCCAATAAAATTAAATCTGAGAAG |
| | ACACACATCCACACCTCCTTCAGTC |
| | ACTTCAACAAATCGAGAGTATGATT |
| | GAAGCTGAGTCCTCTGTCAAGGAGA |
| | AAGACATGACCAAAGAGTTCTTTGA |
| | AAACAAATCAGAAACATGGCCCGTT |
| | GGAGAGTCCCCCAAAGGAGTGGAGG |
| | AAGGTTCCATTGGGAAGGTCTGCAG |
| | AACTTTATTGGCAAAGTCGGTATTC |
| | AACAGCTTGTATGCATCTCCACAAC |
| | TGGAAGGATTTTCAGCTGAATCAAG |
| | AAAACTGCTTCTTATCGTTCAGGCT |
| | CTTAGGGACAACCTGGAACCTGGGA |
| | CCTTTGATCTTGGGGGGCTATATGA |
| | AGCAATTGAGGAGTGCCTGATTAAT |
| | GATCCCTGGGTTTTGCTTAATGCTT |
| | CTTGGTTCAACTCCTTCCTCACACA |
| | TGCATTGAGATAGTTGTGGCAATGC |
| | TACTATTTGCTATCCATACTGTCCA |
| | AAAAAGTACCTTGTTTCTACT |
| | (SEQ ID NO: 16) |
| PB1D4 | AGCGAAAGCAGGCAAACCATTTGAA |
| | TGGATGTCAATCCGACTTTACTTTT |
| | CTTAAAAGTGCCAGCACAAAATGCT |
| | ATAAGCACAACTTTCCCTTATACTG |
| | GAGACCCTCCTTACAGCCATGGGAC |
| | AGGAACAGGATACACCATGGATACT |
| | GTCAACAGGACACATCAGTACTCAG |
| | AAAGGGGAAGATGGACAACAAACAC |
| | CGAAACTGGAGCACCGCAACTCAAC |
| | CCGATTGATGGGCCACTGCCAGAAG |
| | ACAATGAACCAAGTGGTTATGCCCA |
| | AACAGATTGTGTATTGGAAGCAATG |
| | GCCTTCCTTGAGGAATCCCATCCTG |
| | GTATCTTTGAGACCTCGTGTCTTGA |
| | AACGATGGAGGTTGTTCAGCAAACA |
| | CGAGTGGACAAGCTGACACAAGGCC |
| | GACAGACCTATGACTGGACTCTAAA |
| | TAGGAACCAGCCTGCTGCAACAGCG |
| | GATTACCAGGGGCGTTTATGCAACC |
| | CACTGAACCCATTTGTCAACCATAA |
| | AGACATTGAATCAGTGAACAATGCA |
| | GTGATAATGCCAGCACATGGTCCAG |
| | CCAAAAACATGGAGTATGATGCTGT |
| | TGCAACAACACACTCCTGGATCCCC |
| | AAAAGAAATCGATCCATCTTGAATA |
| | CAAGCCAAAGAGGAATACTTGAAGA |
| | TGAACAAATGTACCAAAAGTGCTGC |
| | AACTTATTTGAAAAATTCTTCCCCA |
| | GCAGTTCATACAGAAGACCAGTCGG |
| | GATATCCAGTATGGTGGAGGCTATG |
| | GTTTCCAGAGCCCGAATTGATGCAC |
| | GAATTGATTTCGAATCTGGAAGGAT |
| | AAAGAAAGAGGAGTTCACTGAGATC |
| | ATGAAGATCTGTTCCACCATTGAAG |
| | AGCTCAGACGGCAAAAATAGTGAAT |
| | TTAGCTTGTCCTTCATGAAAAAATG |
| | CCTTGTTTCTACT |
| | (SEQ ID NO: 17) |
| PB2D4 | AGCGAAAGCAGGTCAATTATATTCA |
| | ATATGGAAAGAATAAAAGAACTAAG |
| | GAATCTAATGTCGCAGTCTCGCACT |
| | CGCGAGATACTCACAAAAACCACCG |
| | TGGACCATATGGCCATAATCAAGAA |
| | GTACACATCAGGAAGACAGGAGAAG |
| | AACCCAGCACTTAGGATGAAATGGA |
| | TGATGGCAATGAAATATCCAATTAC |
| | AGCAGACAAGAGGATAACGGAAATG |
| | ATTCCTGAGAGAAATGAGCAGGGAC |
| | AAACTTTATGGAGTAAAATGAATGA |
| | CGCCGGATCAGACCGAGTGATGGTA |
| | TCACCTCTGGCTGTGACATGGTGGA |
| | ATAGGAATGGACCAGTGACAAGTAC |
| | AGTTCATTATCCAAAAATCTACAAA |
| | ACTTATTTTGAAAAAGTCGAAAGGT |
| | TAAAACATGGAACCTTTGGCCCTGT |
| | CCATTTTAGAAACCAAGTCAAAATA |
| | CACTTCTTCCCTTCGCAGCCGCTCC |
| | ACCAAAGCAAAGTGGAATGCAGTTC |
| | TCCTCATTGACTATAAATGTGAGGG |
| | GATCAGGAATGAGAATACTTGTAAG |
| | GGGCAATTCTCCAATATTCAACTAC |
| | AACAAGACCACTAAAAGACTCACAG |
| | TTCTCGGAAAGGATGCTGGCCCTTT |
| | AACTGAAGACCCAGATGAAGGCACA |
| | GCTGGAGTTGAGTCCGCAGTTCTGA |
| | GAGGATTCCTCATTCTGGGCAAAGA |
| | AGACAGGAGATATGGACCAGCATTA |
| | AGCATAAATGAACTGAGCAACCTTG |
| | CGAAAGGAGAGAAGGCTAATGTGCT |
| | AATTGGGCAAGGAGACGTGGTGTTG |
| | GTAATGAAACGGAAACGGAACTCTA |
| | GCATACTTACTGACAGCCAGACAGC |
| | GACCAAAAGAATTCGGATGGCCATC |
| | AATTAGTGTCGAATAGTTTAAAAAC |
| | GACCTTGTTTCTACT |
| | (SEQ ID NO: 18) |
| PAD5 | AGCGAAAGCAGGTACTGATTCAAAA |
| | TGGAAGATTTTGTGCGACAATGCTT |
| | CAATCCGATGATTGTCGAGCTTGCG |
| | GAAAAGGCAATGAAAGAGTATGGAG |
| | AGGACCTGAAAATCGAAACAAACAA |
| | ATTTGCAGCAATATGCACTCACTTG |
| | GAAGTGTGCTTCATGTATTCAGATT |
| | TTCACTTCATCGATGAGCAAGGCGA |
| | GTCAATAGTCGTAGAACTTGGCGAT |
| | CCAAATGCACTTTTGAAGCACAGAT |
| | TTGAAATAATCGAGGGAAGAGATCG |
| | CACAATAGCCTGGACAGTAATAAAC |
| | AGTATTTGCAACACTACAGGGGCTG |
| | AGAAACCAAAGTTTCTACCAGATTT |
| | GTATGATTACAAGAAGAATAGATTC |
| | ATCGAAATTGGAGTAACAAGGAGAG |
| | AAGTTCACATATACTATCTGGAAAA |
| | GGCCAATAAAATTAAATCTGAGAAG |
| | ACACACATCCACATTTTCTCATTCA |
| | CTGGGGAGGAAATGGCCACAAAGGC |
| | CGACTACACTCTCGATGAAGAAAGC |
| | AGGGCTAGGATCAAAACCAGGCTAT |
| | TCACCATAAGACAAGAAATGGCTAG |
| | CAGAGGCCTCTGGGATTCCTTTCGT |
| | CAGTCAATGGGAGAAGTACTGTGTT |
| | CTTGAGGTAGGAGATATGCTTCTAA |
| | GAAGTGCCATAGGCCATGTGTCAAG |
| | GCCTATGTTCTTGTATGTGAGGACA |
| | AATGGAACCTCAAAAATTAAAATGA |
| | AATGGGGGATGGAAATGAGGCGTTG |
| | CCTCCTTCAGTCACTTCAACAAATC |
| | GAGAGTATGATTGAAGCTGAGTCCT |
| | CTGTCAAGGAGAAAGACATGACCAA |
| | AGAGTTCTTTGAAAACAAATCAGAA |
| | ACATGGCCCGTTGGAGAGTCCCCCA |
| | AAGGAGTGGAGGAAGGTTCCATTGG |
| | GAAGGTCTGCAGAACTTTATTGGCA |
| | AAGTCGGTATTCAACAGCTTGTATG |
| | CATCTCCACAACTGGAAGGATTTTC |
| | AGCTGAATCAAGAAAACTGCTTCTT |
| | ATCGTTCAGGCTCTTAGGGACAACC |
| | TGGAACCTGGGACCTTTGATCTTGG |
| | GGGGCTATATGAAGCAATTGAGGAG |
| | TGCCTGATTAATGATCCCTGGGTTT |
| | TGCTTAATGCTTCTTGGTTCAACTC |
| | CTTCCTCACACATGCATTGAGATAG |
| | TTGTGGCAATGCTACTATTTGCTAT |
| | CCATACTGTCCAAAAAAGTACCTTG |
| | TTTCTACT (SEQ ID NO: 19) |

TABLE 4
Primers for DIG Construction

| | | Oligonucleotide sequence (5' to 3') | |
|---|---|---|---|
| Plasmid | Primer | (SEQ ID Nos: 32-82 and 84)) | Restriction |
| | PB2-F | TATTGGTCTCAGGGAGCGAAAGCAGGTC (32) | BsaI |
| PB2D1 | PB2-MR | GGTTCTTCTCCTGTCTTCCTG (33) | |
| | PB2-MF | GAAGTACACATCAGGAAGACAGGAGAAGAACCGGAGAGAAC | (34) |
| | PB2-R | ATATGGTCTCGTATTAGTAGAAACAAGGTCGTTT (35) | BsaI |
| | PB1-F | TATTCGTCTCAGGGAGCAAAAGCAGGCA (36) | BsmBI |
| PB1D1 | PB1-MR | TGACAGTATCCATGGTGTATC (37) | |
| | PBI-MF | GGGACAGGAACAGGATACACCATGGATACTGTCATTCCAGAG | (38) |
| | PB1-R | ATATCGTCTCGTATTAGTAGAAACAAGGCATTT (39) | BsmBI |
| | PA-F | TATTCGTCTCAGGGAGCAAAAGCAGGTAC (40) | BsmBI |
| PAD1 | PA-MR | ACTTCCAAGTGAGTGCATATTG (41) | |
| | PA-MF | ACAAATTTGCAGCAATATGCACTCACTTGGAAGTGGCTATATG | (42) |
| | PAR | ATATCGTCTCGTATTAGTAGAAACAAGGTACTT (43) | BsmBI |
| | PB2-F | TATTGGTCTCAGGGAGCGAAAGCAGGTC (44) | BsaI |
| PB2D2 | PB2-MR | CATTTCCGTTATCCTCTTGTCTG (45) | |
| | PB2-MF | CCAATTACAGCAGACAAGAGGATAACGGAAATGGGGCAAAG | (46) |
| | PB2-R | ATATGGTCTCGTATTAGTAGAAACAAGGTCGTTT (47) | BsaI |
| | PB1-F | TATTCGTCTCAGGGAGCAAAAGCAGGCA (48) | BsmBI |
| PB1D2 | PB1-MR | GTTGAGTTGCGGTGCTCCAGTTTCG (49) | |
| | PBI-MF | CAACAAACACCGAAACTGGAGCACCGCAACTCAACAATTCTT | (50) |
| | PB1-R | ATATCGTCTCGTATTAGTAGAAACAAGGCATTT (51) | BsmBI |
| | PA-F | TATTCGTCTCAGGGAGCAAAAGCAGGTAC (52) | BsmBI |
| PAD2 | PA-MR | ATCGCCAAGTTCTACGACTATTG (53) | |
| | PA-MF | GCAAGGCGAGTCAATAGTCGTAGAACTTGGCGATCAAGAAAA | (54) |
| | PAR | ATATCGTCTCGTATTAGTAGAAACAAGGTACTT (55) | BsmBI |
| | PB2-F | TATTGGTCTCAGGGAGCGAAAGCAGGTC (56) | BsaI |
| PB2D3 | PB2-MR | GGTGATACCATCACTCGGTCTG (57) | |
| | PB2-MF | CGCCGGATCAGACCGAGTGATGGTATCACCTAACTGAAGACC | (58) |
| | PB2-R | ATATGGTCTCGTATTAGTAGAAACAAGGTCGTTT (84) | BsaI |
| | PB1-F | TATTCGTCTCAGGGAGCAAAAGCAGGCA (59) | BsmBI |
| PB1D3 | PB1-MR | ATCTGTTTGGGCATAACCAC (60) | |
| | PBI-MF | ACAATGAACCAAGTGGTTATGCCCAAACAGATCAAAAGAAAT | (61) |
| | PB1-R | ATATCGTCTCGTATTAGTAGAAACAAGGCATTT (62) | BsmBI |
| | PA-F | TATTCGTCTCAGGGAGCAAAAGCAGGTAC (63) | BsmBI |
| PAD3 | PA-MR | TCATACAAATCTGGTAGAAACTTTG (64) | |
| | PA-MF | AGAAACCAAAGTTTCTACCAGATTTGTATGAGAGAGTCCCCCA | (65) |
| | PAR | ATATCGTCTCGTATTAGTAGAAACAAGGTACTT (66) | BsmBI |
| | PB2-F | TATTGGTCTCAGGGAGCGAAAGCAGGTC (67) | BsaI |
| PB2D4 | PB2-MR | GTATTTTGACTTGGTTTCTAA (68) | |
| | PB2-MF | GCCCTGTCCATTTTAGAAACCAAGTCAAAATACACTTCTTCC (69) | |
| | PB2-R | ATATGGTCTCGTATTAGTAGAAACAAGGTCGTTT (70) | BsaI |
| | PB1-F | TATTCGTCTCAGGGAGCAAAAGCAGGCA (71) | BsmBI |
| PB1D4 | PB1-MR | GCTGTTGCAGCAGGCTGGTTC (72) | |
| | PBI-MF | GGACTCTAAATAGGAACCAGCCTGCTGCAACAGCGGATTAC (73) | |
| | PB1-R | ATATCGTCTCGTATTAGTAGAAACAAGGCATTT (74) | BsmB |
| | PA-F | TATTCGTCTCAGGGAGCAAAAGCAGGTAC (75) | BsmBI |
| PAD4 | PA-MR | TGTGGATGTGTGTCTTCTCAG (76) | |
| | PA-MF | CAATAAAATTAAATCTGAGAAGACACACATCCACACCTCCTT (77) | |
| | PAR | ATATCGTCTCGTATTAGTAGAAACAAGGTACTT (78) | BsmB |
| | PA-F | TATTCGTCTCAGGGAGCAAAAGCAGGTAC (79) | BsmBI |
| PAD5 | PA-MR | GACTGACGAAAGGAATCCCAGAG (80) | |
| | PA-MF | CTAGCAGAGGCCTCTGGGATTCCTTTCGTCAGTCAATGGGAG (81) | |
| | PAR | ATATCGTCTCGTATTAGTAGAAACAAGGTACTT (82) | BsmB |

In Vitro DIG Transfection and Antiviral Assay

For in vitro antiviral experiments, plasmids of DI-PA, DI-PB1, DI-PB2, CD1200, CD3600 and empty vector PHW2000 were transfected into A549 or HK-2 cells by Lipofectamine 3000 reagent (Invitrogen, Cat^{#} 1857483) or GeneJuice (Sigma, Cat^{#} 70967) according to the manufacturer's instructions. After 24 hours transfection, the cell transfection media was replaced by fresh DMEM with 0.005 MOI of A(H7N7) virus or 0.01 MOI of SARS-CoV-2 for infection in A549 cells (influenza) or HK-2 cells (SARS-CoV-2). Supernatants were collected at 48 hours post infection. Viral titers were determined using plaque assay as described previously [Zhao, H. et al., Sci Rep 6, 22008, Doi: 10.1038/srep22008 (2016)]. The antiviral activity of DIGs was generated by comparing the viral titers in the supernatants of cells transfected with DIG-plasmids or empty vector PHW.

Toxicity Assay

TAT-P1 and TAT2-P1 were synthesized by ChinaPeptide (Shanghai, China, see Table 5). Cytotoxicity was determined by the detection of 50% cytotoxic concentration (CC₅₀) using a tetrazolium-based colorimetric MTT assay as described previously [Zhao, H. et al., Sci Rep 6, 22008, Doi: 10.1038/srep22008 (2016)]. 293T cells were seeded in 96-well cell culture plate at an initial density of 3×10⁴ cells per well in DMEM supplemented with 10% FBS. After overnight culture, cell culture media were replaced with fresh DMEM supplemented with various concentrations of peptides and 1% FBS. After 24 hours incubation at 37° C., MTT solution (5 mg ml⁻¹, 10 μl per well) was added to each well for incubation at 37° C. for 4 hours, and then 100 μl of 10% SDS in 0.01 M HCl was added to each well. After further incubation at room temperature with shaking overnight, plates were read at OD₅₇₀ using Victor™ X3 Multilabel Reader (PerkinElmer, USA). Cell culture wells without peptides were used as the experiment control and medium only served as a blank control. The in vivo toxicity of nanoparticles was measured by testing the body weight loss of mice intratracheally inoculated with TAT-P1/DNA (20μg/5 μg), TAT2-P1/DNA (20 μg/5 μg), and in vivo jetPEI/DNA (0.7 μl/5 μg).

TABLE 5
Peptide sequences.

| Peptide | Peptide sequence |
|---|---|
| TAT-P1 | YGRKKRRQRRRCWGPCPTAFRQIGNCG |
| | RFRVRCCRIR (SEQ ID NO: 3) |
| TAT2-P1 | RKKRRQRRRCWGPCPTAFRQIGNCGRF |
| | RVRCCRIR (SEQ ID NO: 4) |

Gel Retardation Assay

According to a previous study [Zhao, H. et al. Nat Commun, 9, 2358, Doi: 10.1038/s41467-018-04792-7, (2018)], peptides and DNA were premixed at various weight ratios with 0.5 μg plasmid DNA in 4 μl distilled water at room temperature for 15 mins. The samples were loaded into a 1% w/v agarose gel containing ethidium bromide nucleic acid stain. Gel electrophoresis was performed in TBE buffer at 120 V for 20 min and then the agarose gel was visualized under the ultraviolet (UV) illumination.

Hydrodynamic Particle Size Measurement

According to a previous study [Zhao, H. et al. Nat Commun, 9, 2358, Doi: 10.1038/s41467-018-04792-7, (2018)], peptide and DNA were prepared at various weight ratios. Peptide and plasmid DNA solution were prepared separately in distilled water. Equal volumes of peptide and plasmid DNA solution were mixed together to give a final volume of 4 μl containing 0.5 μg of plasmid DNA. After incubation of the complexes at room temperature for 15 min, the 4 μl complexes were diluted to 50 μl in distilled water, and then the particle diameters were measured by DynaPro® Plate Reader (Wyatt, USA).

Transmission Electron Microscopy Assay

To determine virus or nanoparticle size and shape, virus or peptide/DNA nanoparticles premixed with the weight ratio (4:1) of peptide:DNA were applied to continuous carbon grids and excess solution was removed. The grids were transferred into 4% uranyl acetate and incubated for 1 minute. After removing the solution, the grids were air-dried at room temperature [Zhao, H. et al., Nat Commun 12, 1517, Doi: 10.1038/s41467-021-21825-w (2021)]. For each peptide/DNA nanoparticle, two independent experiments were done for taking TEM images by FEI Tecnal G2-20 TEM.

In Vitro Luminescence Assay

Peptides and plasmid DNA were mixed at various weight ratios with 0.5 μg plasmid DNA in 4μl distilled water. After 15 min incubation at room temperature, 293T cells in 24-well plate were transfected with the peptide/DNA complexes including peptides and 0.1 μg of each pHW2000 plasmid encoding the PA, PB1, PB2, NP, and the mini-genome of pPoLI-fluc-RT (pLuciferase, the firefly luciferase reporter) [Zhao, H. et al. Virology 498, 1-8, Doi: 10.1016/j.virol.2016.08.004 (2016)]. After 24 hours transfection, luminescence was measured by Luciferase assay system (Promega, Cat^{# E}1910) with a Victor X3 Multilabel reader (PerkinElmer, USA). The luminescence reading was normalized to 1 mg protein.

In Vivo Bioluminescence Analysis

Peptides with pCMV-Cypridina Luc (pCMV-Luc, ThermoFisher, Cat^{# RF}233236) complexes were prepared at weight ratios (4:1) of peptide:DNA with 5 μg plasmid DNA in 40 μl distilled water. After leaving the complexes for 15 minutes at room temperature, the complexes were intratracheally inoculated to mouse lungs at 24 hours before measuring the luciferase expression in lung tissues. The jetPEI/pCMV-Luc (0.7 μl/5.0 μg) complexes were prepared according to the manufacturer's protocol as a positive control (Polyplus Transfection, Cat^{#} 201-10G). Mouse lungs inoculated with peptide or jetPEI only were used as the negative control. For detecting bioluminescence signal, mouse lung tissues were homogenized and centrifuged at 15,000×g for 10 min The supernatants were used to analyze the luciferase protein expression by Cypridina luciferase flash assay kit (ThermoFisher, Cat^{#} 16168). The luciferase expression level in mouse lungs was normalized to 1 mg protein and TAT-P1 which was set as 1000 as the normalized reference for the analysis. For in vivo bioluminescence imaging, mouse lungs were taken out and then substrate was added to lungs for taking image by IVIS® Spectrum In Vivo Imaging System (PerkinElmer, USA).

RT-qPCR Assay

Real-time RT-qPCR was performed as described previously [Zhao, H. et al., Sci Rep 6, 22008, Doi: 10.1038/srep22008 (2016)]. RNA was reverse transcribed to cDNA using primer Uni-12 or random primers by PrimeScript II 1st Strand cDNA synthesis Kit (Takara, Cat^{#} 6210A) using GeneAmp® PCR system 9700 (Applied Biosystems, USA). The cDNA was then amplified using specific primers for DI-PA, DI-PB1, DI-PB2, cytokines and chemokines using LightCycle® 480 SYBR Green I Master (Roach, USA, see Table 3). For quantitation, 10-fold serial dilutions of standard plasmid equivalent to 10¹ to 10⁶ copies per reaction were prepared to generate the calibration curve. Real-time qPCR experiments were performed using LightCycler® 96 system (Roche, USA).

In Vivo Antiviral Assay

Antiviral analysis of nanoparticle TAT-P1/DIG and TAT2-P1/DIG in vivo was carried out in BALB/c mice and hamsters [Zhao, H. et al., Nat Commun 12, 1517, Doi: 10.1038/s41467-021-21825-w (2021)]. BALB/c female mice (10-12 weeks for H1N1 virus) were obtained from The University of Hong Kong Centre for Comparative Medicine Research and female hamsters (4-6 weeks for SARS-CoV-2) were obtained from the Chinese University of Hong Kong Laboratory Animal Services Centre through The University of Hong Kong Centre for Comparative Medicine Research. Animals were kept in biosafety level 2/3 laboratory (housing temperature between 22-25° C. with dark/light cycle) and given access to standard pellet feed and water ad libitum. All experimental protocols followed the standard operating procedures of the approved biosafety level 2 animal facilities and were approved by the Committee on the Use of Live Animals in Teaching and Research of the University of Hong Kong [Zheng, B. J. et al., Proceedings of the National Academy of Sciences of the United States of America 105, 8091-8096, Doi: 10.1073/pnas.0711942105 (2008)]. The mouse adapted A(H1N1)pdm09 virus was used for lethal challenge in mice. To evaluate the prophylactic efficacy, mice were intratracheally inoculated with 40 μl of TAT-P1/DIG (20.0 μg/5.0 μg), TAT2-P1/DIG (20.0 μg/5.0 μg), or zanamivir (40.0 μg/mouse in PBS), 1-10 days before viral challenge. Flu vaccine-480 (480.0 ng in PBS) was intramuscularly injected into the mice. Next, the mice were intranasally inoculated with 3 LD50 of virus. Survivals and general conditions were monitored for 16 days or until death. For DIG against SARS-CoV-2 (B.1.617.2, Delta) in hamsters, TAT2-P1/CD3600 or TAT2-P1/PHW (50.0 μg/12.5 μg) were inoculated in hamster lungs at day 1 or day 3 before viral inoculation (500 PFU). Experimental animals were randomly allocated to each group. Viral loads and histopathology changes in hamster lungs were tested at day two post-infection.

### Results

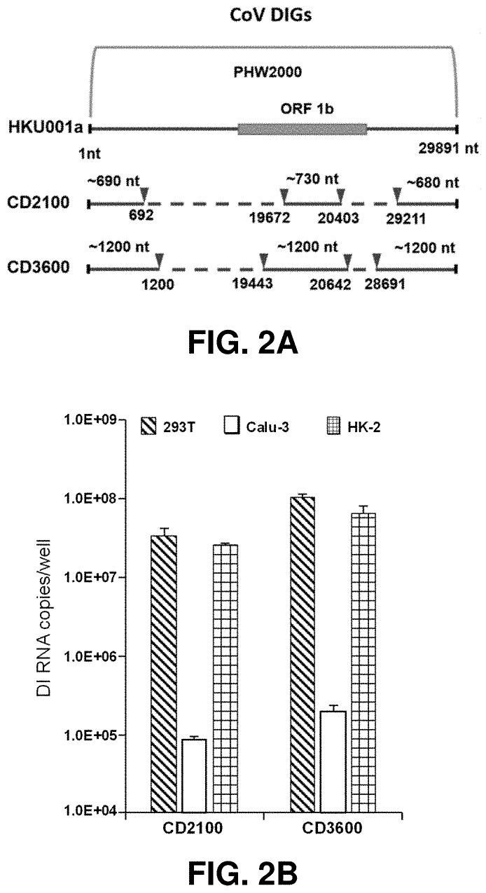

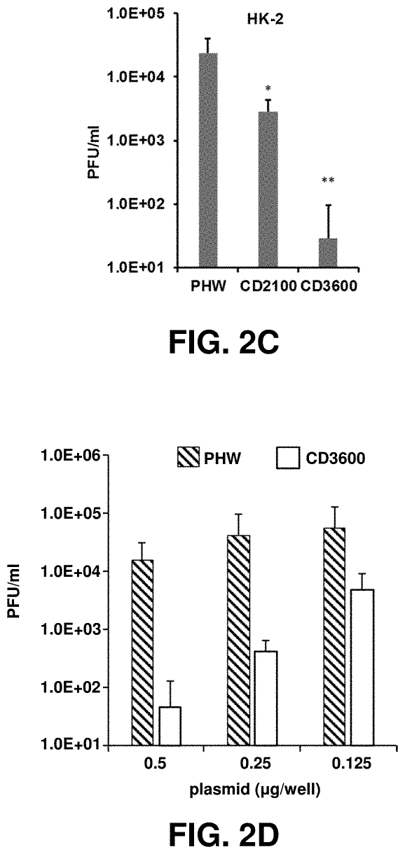

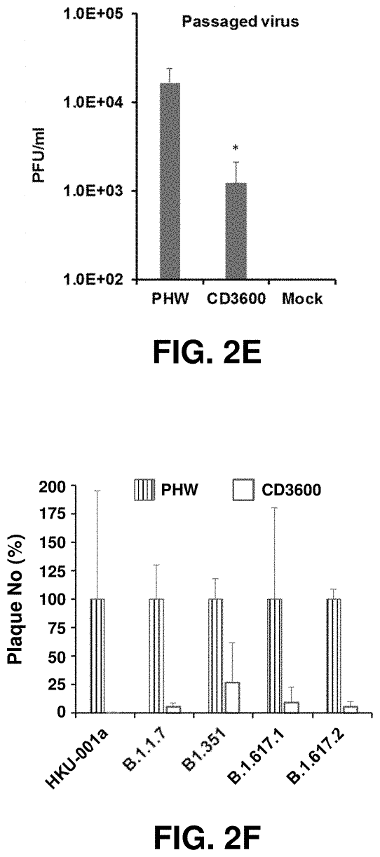

Coronavirus DIG inhibited SARS-CoV-2 replication in human HK-2 cells So far, there are limited studies to show if defective genes of coronavirus could significantly interfere with coronavirus replication [Vignuzzi, M. & Lopez, C. B. Nat Microbiol 4, 1075-1087, Doi: 0.1038/s41564-019-0465-y (2019)]. To identify DIG against coronavirus, according to the possible packaging sequences of SARS-CoV [Qin, L. et al., Acta Pharmacol Sin 24, 489-496, (2003); Masters, P. S. Virology 537, 198-207, Doi: 10.1016/j.virol.2019.08.031 (2019)], coronavirus DIGs (CD2100 and CD3600) which included the 5′-end sequences, internal sequences in ORF 1b and the 3′-end sequences of SARS-CoV-2 were constructed (FIG. 2A). Gene expression was first tested in human cell lines including 293T, Calu-3 and HK-2. High RNA levels of CD2100 and CD3600 were demonstrated in transfected HK-2 cell lines similar to the RNA expression in 293T cells (FIG. 2B). Thus, HK-2 was selected for the antiviral assay of CD2100 and CD3600. After DIG transfection in HK-2 cells, SARS-CoV-2 was added to cells for infection. CD3600 and CD2100 could significantly inhibit SARS-CoV-2 replication when compared with empty vector PHW (FIG. 2C). CD3600 showed significantly better antiviral activity than that of CD2100. To further confirm the antiviral efficiency of CD3600 in cells, it was demonstrated that CD3600 could significantly inhibit SARS-CoV-2 replication in a dose-dependent manner when compared with PHW (FIG. 2D).

The capacity of CoV-DIG to exert antiviral activity in multiple growth cycles which may contribute to the expanded antiviral activity of DIGs was tested. It was observed that virus in supernatants of cells with CD3600 showed significantly less viral replication at 24 hours post-infection during viral passages in VeroE6/TMPRSS2 cells when compared with non-DIG vector (PHW) controls (FIG. 2E). This indicated that transfected CD3600 could exert extended activity against SARS-CoV-2 replication even during viral passages in multiple growth cycles in VeroE6/TMPRSS2 cells. To confirm the presence of CD3600 defective virus in passaged supernatants in VeroE6/TMPRSS2 cells, the same PFU of PHW-treated virus and CD3600-treated virus was cultured and the supernatants were collected at 72 hours post-infection for ultracentrifugation to recover the virus for TEM assay. CD3600-treated cell culture supernatant demonstrated increased small spherical particles (<40 nm) when compared with PHW-treated cell culture supernatant (100-150 nm). This result may indicate that treatment with CD3600 led to the packaging of smaller defective viral particles. Furthermore, RT-qPCR data revealed high CD3600 RNA copies in the supernatants but lower PFU titer when compared with PHW-treated virus. Moreover, broad antiviral activity of CD3600 against five other SARS-CoV-2 variants was demonstrated, consistent with the broad antiviral mechanism of DIG involving consuming the viral replication and packaging components of viral replication, which competitively inhibits viral packaging without targeting a specific viral protein. In conclusion, these results indicated that CoV-DIG with the 5′-end, internal sequence in ORF 1b, and 3′-end of SARS-Cov-2 could broadly inhibit SARS-CoV-2 replication by maintaining its self-sustaining DIG production in multiple growth cycles.

The Optimal Defective Interfering Genes for Anti-Influenza Virus in A549 Cells and in Mice

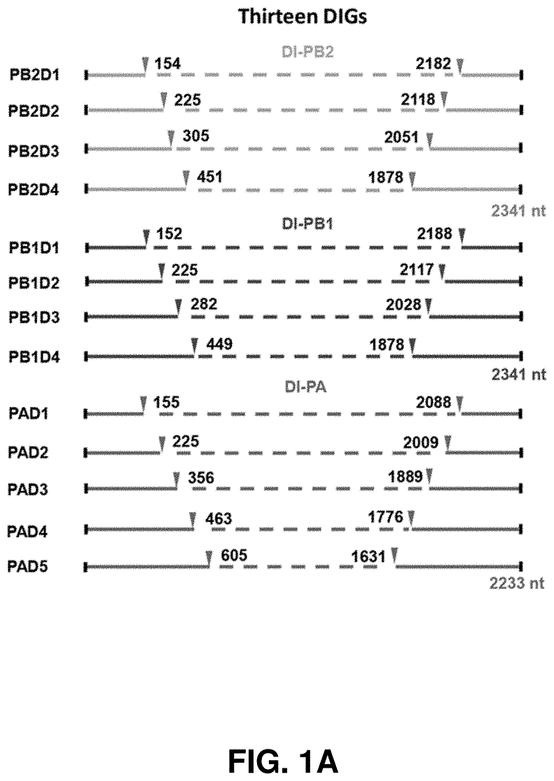

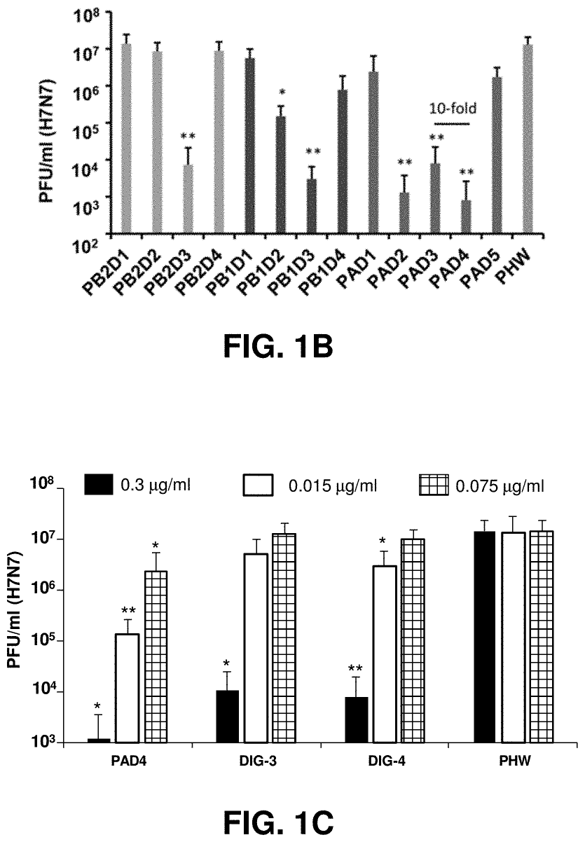

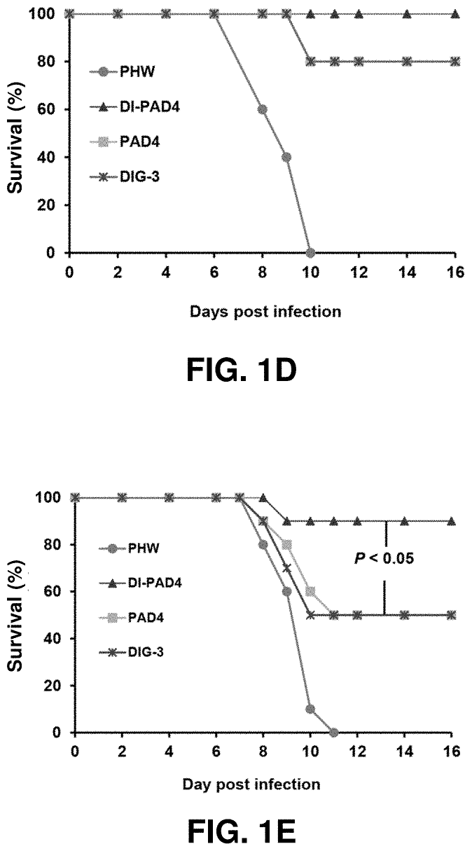

To identify the potent antiviral DIG against influenza virus, ten DIGs with diverse gene lengths at the 3′ and 5′ ends of polymerase genes ranging from 150 nt to 600 nt (FIG. 1A) were designed and constructed to identify DIGs having more potent antiviral activity in vitro and in vivo. Each DIG was transfected into A549 cells and then cells were infected with A(H7N7) virus at 24 hours after transfection without the need of adding trypsin to the cell culture (FIG. 1B). PAD4, which contains 450 nt at the 3′ and 5′ end of the PA gene segment, exhibited the most potent antiviral activity with 3-4 log reduction of viral load, which was (4-10)-fold lower than that of PAD3, PB1D3 and PB2D4 used in a previous study [Zhao, H. et al. Nat Commun, 9, 2358, Doi: 10.1038/s41467-018-04792-7, (2018)]. PB2D3, PB1D3, PAD2, and PAD3 showed very potent antiviral activity with 2-3 log inhibition of viral replication. PB2D2, PB2D4, PB1D2, PB1D4, and PADS showed weak antiviral activity with or without significant difference (FIG. 1B). PB2D1, PB1D1, and PAD1 with ˜150 nt at the 3′ and 5′ ends did not show antiviral activity, which is consistent with the fact that the polymerase genes need at least 150 nt at the 5′ end for viral gene packaging, as demonstrated by previous studies [Duhaut, S. D. & Dimmock, N. J. J Gen Virol 83, 403-411, Doi: 10.1099/0022-1317-83-2-403. (2002); Duhaut, S. D. & Dimmock, N. J. Virology 248, 241-253, Doi: 10.1006/viro.1998.9267 (1998)]. These data indicated that PAD4 with 450 nt at the 3′ and 5′ ends showed the best antiviral activity. PB2D3 with 300 nt and PB1D3 with 300 nt at the 3′ and 5′ ends, showed better antiviral activity than DIGs that were too short or too long, respectively. Next, the antiviral activity of single PAD4 was further tested and compared with two combinatorial DIGs (FIG. 1C) in different concentrations. PAD4 alone showed more potent antiviral activity with >5-fold lower viral load than that of DIG-3 (the combination of PAD3, PB2D3, and PB1D3) or DI-PAD4 (the combination of PAD4, PB2D3, and PB1D3) in a dose-dependent manner

Considering that multiple DIGs will have more chances to interfere with multiple cognate full-length genes in vivo, DI-PAD4, single PAD4, and DIG-3 were selected to evaluate the antiviral efficacy in mice. Theoretically, three DIGs can generate seven types of defective interfering influenza viruses, which might have increased chances to inhibit wild-type virus replication in mouse lungs than that of single DIG. To transfect DIGs in vivo, DIGs packaged by TAT-P1 vector were intratracheally inoculated into mouse lungs. Two doses of DI-PAD4, single PAD4, or DIG-3, given at 48 hours and 24 hours prior to infection, could protect mice from lethal challenge of A(H1N1)pdm09 virus at 100%, 80%, and 80%, respectively (FIG. 1D), which was significantly better than that of empty plasmid PHW (0%). For 1-dose regimen given intratracheally at 24 hours prior to infection (FIG. 1E), the survival rate of mice receiving DI-PAD4 (90%) was significantly higher than those receiving single PAD4 (50%) or DIG-3 (50%). The survival data indicated that combinational DI-PAD4 can provide better protection than the single PAD4 or DIG-3 in mice. Thus, DI-PAD4 was selected for further studies in vivo.

TAT2-P1 Showed Gene Delivery Efficiency in Lung Tissues

In order to show better antiviral activity of DIGs in vivo, the in vivo delivery efficiency of TAT-peptide based vector was explored. Previous studies indicated that TAT2 (RKKRRQRRR; SEQ ID NO:1) is a shorter form of the peptide which showed better transduction efficiency than that of TAT (YGRKKRRQRRR; SEQ ID NO:2) in vitro [Park, J. et al. J Gen Virol 83, 1173-1181, doi: 10.1099/0022-1317-83-5-1173 (2002); Vives, E., Brodin, P. & Lebleu, B. J Biol Chem 272, 16010-16017, doi: 10.1074/jbc.272.25.16010 (1997)]. Therefore, a TAT2-P1 vector was constructed for evaluating in vitro and in vivo transfection efficiency when compared with TAT-P1.

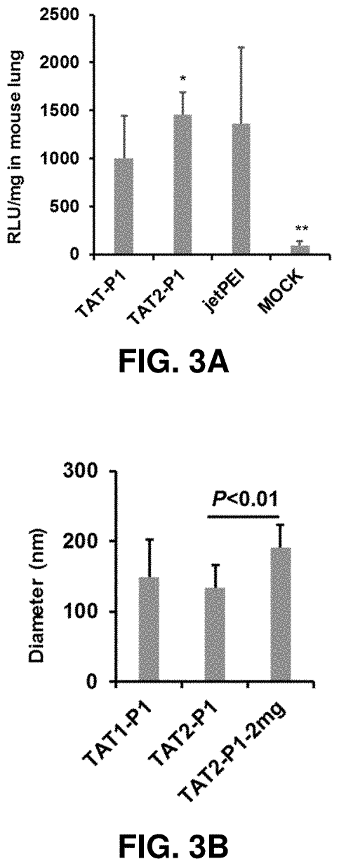

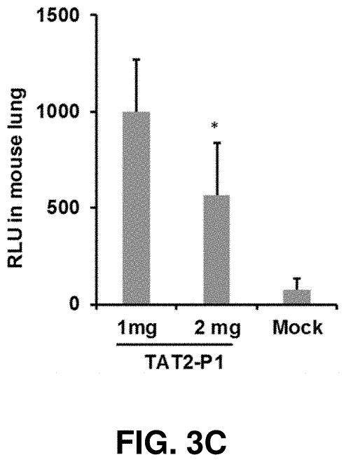

First, DNA binding ability of these peptide-based vectors were determined using gel retardation assay. When the weight ratio of the peptide to plasmid DNA was more than 2, the plasmid DNA could be completely packaged by these vectors. The transfection efficiency showed that the weight ratio (4:1) of peptide:DNA could show effective transfection in cells. Next, to assess the DNA transfection efficiency of these vectors in vivo, pCMV-Luc packaged by TAT-P1, TAT2-P1 or in vivo jetPEI was intratracheally inoculated into mice, and the luciferase expression was measured at 24 h post transfection. TAT2-P1 showed significantly better transfection efficiency in mouse lungs than that of TAT-P1 (FIG. 3A). The toxicity of TAT2-P1 was lower than that of TAT-P1 in cells and in mice.

To identify the mechanism of better transfection efficiency of TAT2-P1 in vivo, the particle sizes of the peptide/pCMV-Luc particles was measured. The pCMV-Luc packaged by TAT-P1 and TAT2-P1 could form particles with a mean diameter of 148 nm and 134 nm, respectively (FIG. 3B). TEM pictures of these nanoparticles showed that the particles formed by TAT2-P1 were uniform spherical nanoparticles and smaller than that of TAT-P1 nanoparticles. These data indicated that the high transfection efficiency of TAT2-P1 in mouse lungs might be related to the smaller particle sizes formed by TAT2-P1 despite the lack of statistical significance.

To confirm that particle size affected the gene delivery of TAT2-P1 in lungs, TAT2-P1/pCMV-Luc was made with different sizes by mixing TAT2-P1 with DNA in different concentrations but with the same amount of peptide/DNA and ratio (4:1). The particle size of TAT2-P1/pCMV-Luc (2 mg ml⁻¹/0.5 mg ml⁻¹) was bigger (˜190 nm) than that (˜130 nm) of TAT2-P1/pCMV-Luc (1 mg ml⁻¹/0.25 mg ml⁻¹). Furthermore, pCMV-Luc packaged by TAT2-P1 (2 mg ml⁻¹) showed significantly lower transfection efficiency than that of pCMV-Luc packaged by TAT2-P1 (1 mg ml⁻¹) in mouse lungs (FIG. 3C), but the transfection efficiency of TAT2-P1 (2 mg ml⁻¹) was unaffected in 293T cells when compared with TAT2-P1 (1 mg ml⁻¹). This result in 293T cells indicated that the lower transfection efficiency of plasmid DNA packaged by TAT2-P1 (2 mg ml⁻¹) than that of TAT2-P1 (1 mg ml⁻¹) in mouse lungs was not due to the difference of DNA package efficiency in the two conditions. Hence, only the difference in particle sizes of nanoparticle TAT2-P1/DNA could significantly affect the gene delivery of TAT2-P1 in mouse lungs. These data indicated that TAT2-P1/DNA could form uniform spherical nanoparticles less than 140 nm, which would cross the barrier of the average pore sizes (100-200 nm) in airway mucus so as to increase DNA uptakes in lungs.

Prophylactic Activity of DIG Against Influenza Virus and SARS-CoV-2

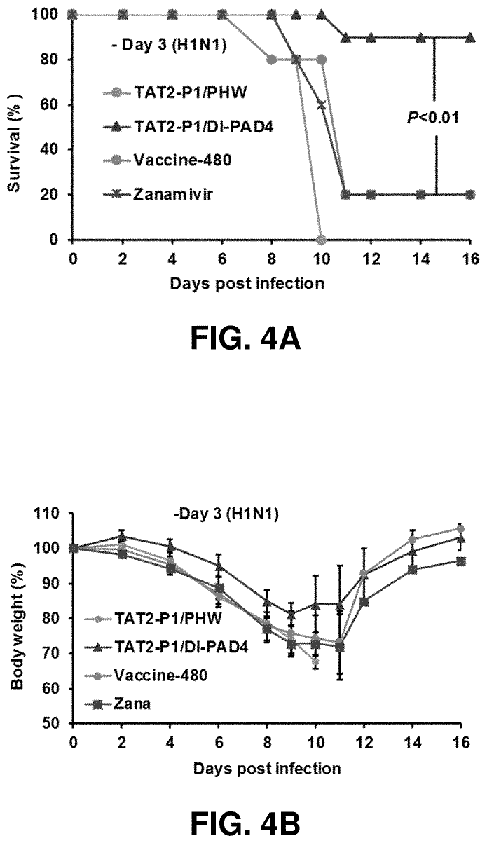

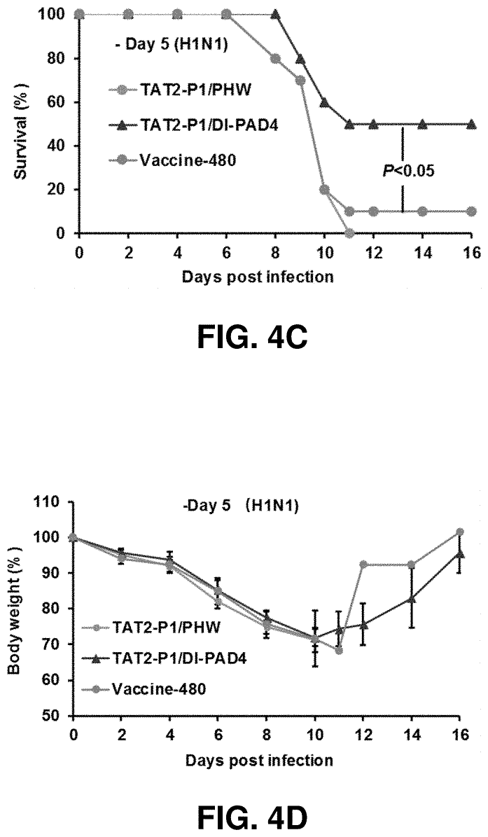

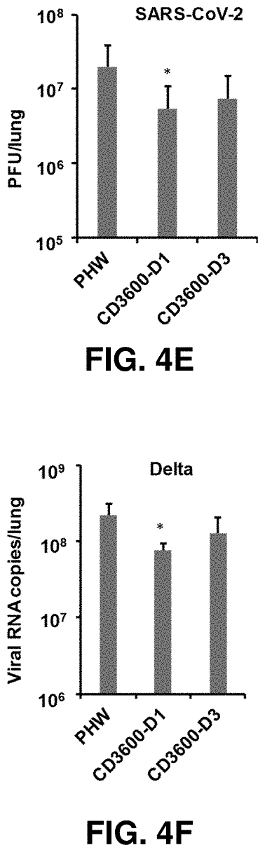

To investigate the prophylactic protection of DIG in vivo, a single dose of TAT2-P1/DIG-4 was used to assess the prophylactic protection on influenza-infected mice when compared with that of vaccine (Influsplit Tetra) and zanamivir [Zhao, H. et al. Nat Commun 9, 2358, doi: Nat Commun (2018)]. Vaccine doses (480 ng) was almost 80-fold higher than that used in humans when using body weight as the reference [Groves, H. T. et al. Front Immunol 9, 126, doi: 10.3389/fimmu.2018.00126 (2018)]. The survival rate (90%) of mice administered TAT2-P1/DI-PAD4 three days prior to infection was significantly better than that of mice administrated with vaccine-480 (20%), zanamivir (20%), or TAT2-P1/PHW (0%, negative control) three days prior to infection (FIG. 4A). Significantly less body weight loss was observed in TAT2-P1/DI-PAD4 treated mice from day 2 to day 8 when compared with TAT2-P1/PHW group (FIG. 4B). The survival rate of mice given zanamivir one day before viral infection was 90%. The effective protection of zanamivir given one day prior to infection and no significant protection given three days prior to infection were consistent with the short half-life time of zanamivir. For mice treated with TAT2-P1/DIG-4 five days prior to viral challenge (FIG. 4C), the survival rate (50%) was significantly higher than that of mice treated with vaccine-480 (10%) or TAT2-P1/PHW (0%). Less body weight loss was observed in TAT2-P1/DIG-4 treated mice from day 4 to day 8 when compared with PHW-treated mice (FIG. 4D). Vaccine-480 showed 40% mouse survival when the given to mice 10 days before viral challenge, which is consistent with the fact that the vaccine needs more than one-two weeks to induce immune protection.

To investigate the antiviral activity of coronavirus CD3600 against SARS-CoV-2 in vivo, TAT2-P1/CD3600 was inoculated into hamster lungs at day 1 or day 3 before SARS-CoV-2 challenge. Because of the non-lethal model of hamsters for SARS-CoV-2 and peak viral titers at day two post-infection in hamster lungs [Chan, J. F. et al. Clin Infect Dis, 71(9):2428-2446. doi: 10.1093/cid/ciaa325 (2020); Kaptein, S. J. F. et al. Proc Natl Acad Sci U S A 117, 26955-26965 doi: 10.1073/pnas.2014441117 (2020)], viral loads in hamster lungs were measured by plaque assay (FIG. 4E) and RT-qPCR (FIG. 4F) at day 2 post-infection. CD3600 significantly inhibited SARS-CoV-2 replication in lungs when given to hamster lungs one day before viral challenge. Reduced inflammation changes in hamster lungs treated by CD3600 were detected when compared with PHW-treated lungs. In conclusion, these in vivo data indicated that one dose of TAT2-P1/DIG could provide rapid-onset (3 or 5 days) prophylactic protection on mice against influenza A virus and could also significantly inhibit SARS-CoV-2 replication in hamsters when DIG was administrated to hamster lungs one day before viral challenge.

TAT2-P1&LAH4 showed high-efficiency gene transfection in lung tissues

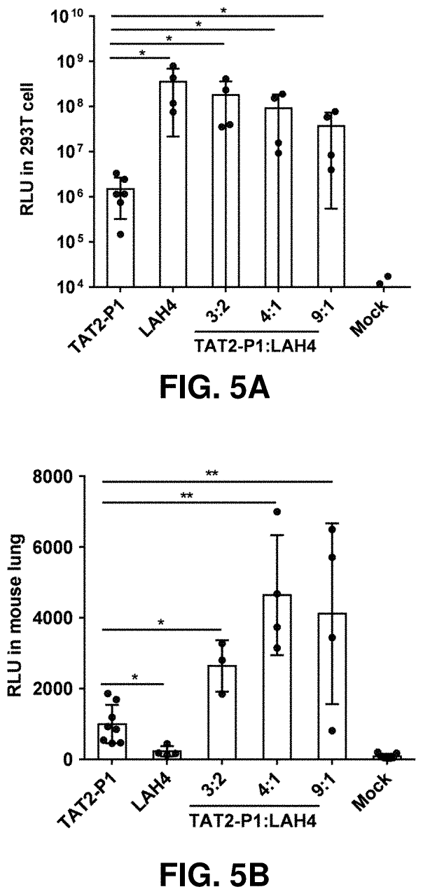

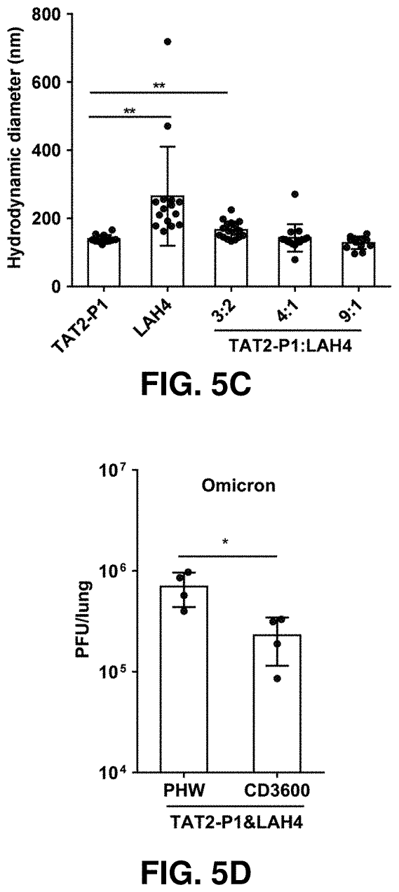

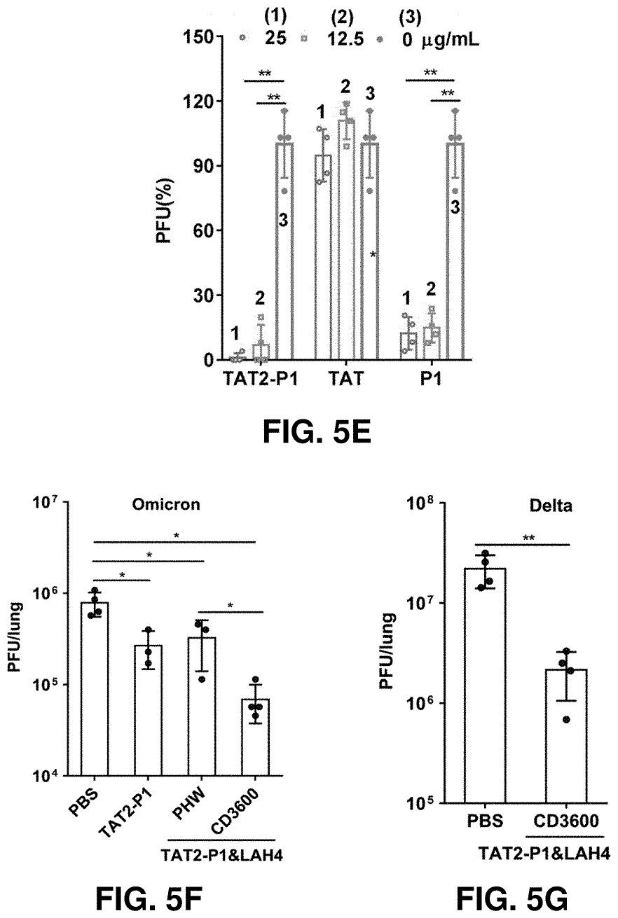

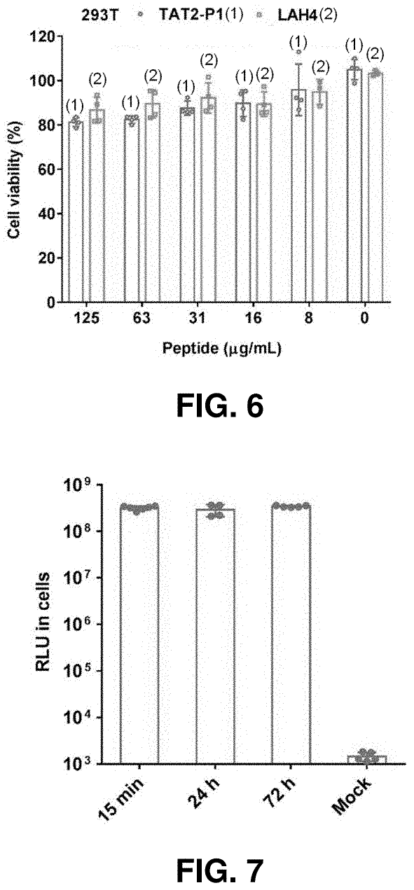

To further investigate peptide-based vectors for gene delivery in the lung airway, it was demonstrated that peptide LAH4 (Lam, J. K. et al. Effective endogenous gene silencing mediated by pH responsive peptides proceeds via multiple pathways. J Control Release 158, 293-303 (2012))showed significantly higher transfection efficiency than TAT2-P1 in cells (FIG. 5A) with TC₅₀ higher than 125 μg ml⁻¹ (FIG. 6), but significantly lower transfection efficiency than TAT2-P1 in lung tissues (FIG. 5B). The high transfection efficiency in cells indicated a high endosomal escape ability of LAH4-packaged plasmids³⁵. However, the low transfection efficiency in lung tissues might be due to the large size of LAH4-nanoparticles (FIG. 5C) which were not spherical in TEM pictures. The pCMV was packaged by LAH4 (1 mg ml-1), TAT2-P1&LAH4 (3:2), TAT2-P1&LAH4 (4:1), and TAT2-P1&LAH4 (9:1). Nanoparticles were negatively stained for Transmission Electron Microscopy analysis. Analysis of TEM pictures revealed that TAT2-P1 and LAH4 could form small spherical nanoparticles. . Because the size and the shape is a potential barrier for passing the mucosal layer in lung tissues (Zhao, F. et al. Cellular uptake, intracellular trafficking, and cytotoxicity of nanomaterials. Small 7, 1322-1337 (2011); Duncan, G. A., et al. The Mucus Barrier to Inhaled Gene Therapy. Mol Ther 24, 2043-2053 (2016); Wang, Z., et al. Size and dynamics of caveolae studied using nanoparticles in living endothelial cells. ACS Nano 3, 4110-4116), it was hypothesized that increasing the transfection efficiency can be accomplished using TAT2-P1 plus LAH4 which could form the small spherical nanoparticles with high endosomal escape ability. When the ratio of TAT2-P1:LAH4 was 4:1 (FIG. 5C), TAT2-P1&LAH4-pCMV could form spherical nanoparticles (˜140 nm). The transfection efficiency (4:1) was significantly higher than that of TAT2-P1 in cells (FIG. 5A) and the TAT2-P1&LAH4 nanoparticles could be stable in room temperature for more than 72 hours (FIG. 7). Importantly, TAT2-P1&LAH4 showed significantly higher transfection efficacy in mouse lungs than that of TAT2-P1 or LAH4 only (FIG. 5B). These results further demonstrated that small nanoparticle sizes (TAT2-P1&LAH4) are important for penetrating airway mucus to increase gene expression in lungs.

TAT2-P1 &LAH4/CD3600 Inhibited SARS-CoV-2 Variants in Hamsters

It was demonstrated that TAT2-P1&LAH4/CD3600 inhibited the replication of SARS-CoV-2 (Omicron) in hamster lungs when one dose was given to hamsters at one day before viral challenge (FIG. 4D). It was shown that defensin-derived peptide P9R could inhibit SARS-CoV-2 (Zhao, H. et al. A broad-spectrum virus- and host-targeting peptide against respiratory viruses including influenza virus and SARS-CoV-2. Nat Commun 11, 4252 (2020)). P1 is derived from P9R (Table 4). It was further demonstrated that P1 and TAT2-P1 significantly inhibited SARS-CoV-2 replication (FIG. 5E). When two doses of TAT2-P1&LAH4/CD3600 were given to hamster lungs at 1-day before and 8-hour after SARS-CoV-2 challenge, TAT2-P1&LAH4/CD3600 more effectively inhibited SARS-CoV-2 Omicron replication in hamster lungs (FIG. 5F). TAT2-P1&LAH4-PHW also inhibited the replication of SARS-CoV-2 (FIG. 5G), which could be attributed to the antiviral activity of TAT2-P1 against SARS-CoV-2 (FIGS. 5E and 5F). The dual function of TAT2-P1 (gene-delivery and antiviral) conferred TAT2-P1&LAH4/CD3600 to show more potent antiviral activity against SARS-CoV-2 in vivo. Moreover, it was demonstrated that two doses of TAT2-P1&LAH4/CD3600 significantly inhibited the replication of Delta SARS-CoV-2 in hamster lungs (FIG. 5F). Nanoparticle staining of hamster lungs was conducted. Hamsters were infected with either the Delta variant or the Omicron variant, and treated with two doses of PBS or TAT2-P1&LAH4/CD3600. The infected lungs were collected at day 2 post infection for NP staining. Staining of tissues non-infected hamster lungs were used as control. The nanoparticle staining further indicated that CD3600 could reduce Omicron and Delta variant replication in hamster lungs. These results indicated that peptide TAT2-P1&LAH4 nanoparticles with the advantages of small spherical particles and high efficiency of endosomal escape ability could effectively penetrate airway mucus to express genes in the lung airway and deliver DIGs to significantly inhibit the replication of SARS-CoV-2 variants in hamster lungs.

Discussion

In this study, it was demonstrated that defective interfering genes (DIG) are useful for inhibiting both influenza virus and coronavirus in vitro and in vivo. CoV-DIG identified in this study significantly inhibit SARS-CoV-2 replication and the inhibition of influenza virus by DIGs may serve as broad-spectrum antivirals with a high barrier to drug resistance. It is contemplated that more DIGs could be discovered for other viruses (including both segmented and non-segmented RNA viruses). With the development of in vivo gene transfection vectors, DIG antivirals may play an increasing role in antiviral development.

For respiratory virus diseases, vaccination is the most effective strategy for prevention. Short of vaccination, the early initiation of antiviral treatment is important for favorable outcomes in patients, which is clearly shown in COVID-19 and influenza virus associated diseases. Currently, prophylactic drugs for pre-exposure and post-exposure of influenza virus are recommended by the World Health Organization (WHO) for daily administration for 10 days despite the concern of drug resistance. No widely available and effective drug is yet recommended for the prophylaxis of coronavirus [Principi, N. et al. Front Med (Lausanne) 6, 109 doi: 10.3389/fmed.2019.00109 (2019); Anand, U. et al. Front Immunol 12, 658519 doi: 10.3389/fimmu.2021.658519. (2021)]. Thus, it is important to identify antivirals with new antiviral mechanism as prophylactic use, which should have the characteristics of rapid-onset, long-acting activity and low possibility to induce drug resistant virus. For influenza virus, one dose of DIG-4 was significantly better than one dose of vaccine for prophylactic protection within 5 days in vivo. The weak prophylactic protection of vaccine on mice within one week was due to the slow onset of vaccine-induced immunity which usually needs around 2 weeks [Groves, H. T. et al. Front Immunol 9, 126 doi: 10.3389/fimmu.2018.00126 (2018); Mohn, K. G et al. Hum Vaccin Immunother 14, 571-578 doi: 10.1080/21645515.2017.1377376. (2018)]. The prophylactic effect of zanamivir indicated that one dose could efficiently protect mice from infection for prophylaxis given one day prior to viral challenge. However, one dose of zanamivir could not provide the same protection as the long-acting DIG-4 in the current experiments.

SARS-CoV-2 vaccines have been found to be promising in controlling COVID-19 in many countries. Due to vaccine hesitancy and variants of concern, antivirals SARS-CoV-2 are also needed as an alternative approach for the better control of coronavirus diseases and reduction of mortality. This study reports the development of CoV-DIG, a different class of antiviral which may complement current anti-coronavirus drug candidates. One dose of coronavirus CD3600 showed rapid-onset of prophylactic activity with marked inhibition of SARS-CoV-2 replication in hamsters. The shorter CD2100 showed less antiviral activity than CD3600 against SARS-CoV-2, and PAD4 showed the most potent antiviral activity against influenza in vitro, which indicates that the length of DIG may determine the effectiveness of blocking wild-type viral replication [Marriott, A. C. & Dimmock, N. J. Rev Med Virol 20, 51-62 doi: 10.1002/rmv.641. (2010)]. The antiviral activity of DIG was not dependent on specific inhibition of a certain protein or gene, but on the inhibition of viral genome packaging into new virions [Meng, B. et al. Virology journal 14, 138 doi: 10.1186/s12985-017-0805-6. (2017)]. This kind of inhibition may be less likely to generate drug resistant viruses when used as prophylactic antivirals.

Previous studies showed that influenza defective interfering virus did not rely on interferon response to protect influenza challenged mice and could protect elderly mice [Scott, P. D., et al. Virology journal 8, 212 (2011); Easton, A. J. et al. Vaccine 29, 2777-2784 doi: 10.1016/j.vaccine.2011.01.102. (2011)]. The rapid-onset of prophylactic antiviral activity of TAT2-P1/DIG in susceptible mice challenged by influenza A virus or hamsters challenged by SARS-CoV-2 also indicated that the antiviral activity might not depend on host immune responses. This may provide additional or potential advantage of DIG for efficient protection in the elderly or the immunocompromised patients. The broad-spectrum antiviral activity of influenza DIG against different influenza viruses and CoV-DIG against SARS-CoV-2 variants suggest that the DIG approach may be less prone to develop viral resistance [Zhao, F. et al. Small 7, 1322-1337 doi: 10.1002/smll.201100001. (2011); Dimmock, N. J. & Easton, A. J. Journal of virology 88, 5217-5227 doi: 10.1128/JVI.03193-13 (2014)]. While influenza and COVID-19 vaccines are less effective among immunosuppressed patients, TAT2-P1/DIG nanoparticle may be a potential candidate for providing rapid-onset prophylactic protection with low possibility of inducing drug resistance against coronavirus and influenza virus.

It is understood that the disclosed methods and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a DIG is disclosed and discussed and a number of modifications that can be made to a number of molecules including the DIG are discussed, each and every combination and permutation of DIG and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a ,” “an,” and “the” include plural reference unless the context clearly dictates otherwise. Thus, for example, reference to “a DIG” includes a plurality of such DIGs, reference to “the DIG” is a reference to one or more DIGs and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different DIGs does not indicate that the listed DIGs are obvious one to the other, nor is it an admission of equivalence or obviousness.

Every polynucleotide disclosed herein is intended to be and should be considered to be specifically disclosed herein. Further, every subgroup that can be identified within this disclosure is intended to be and should be considered to be specifically disclosed herein. As a result, it is specifically contemplated that any polynucleotide, or subgroup of polynucleotides can be either specifically included for or excluded from use or included in or excluded from a list of polynucleotides.

Those skilled in the art will recognize, or be able to ascertain, using no more than routine experimentation, many equivalents to the specific embodiments of the method and compositions described herein. Such equivalents are intended to be encompassed by the following claims.

We claim:

1. An isolated polynucleotide comprising one or more defective interfering genes, wherein each of the one or more defective interfering genes comprises a nucleotide sequence corresponding to one or more portions of a viral gene, wherein the portion of the viral gene comprises a deletion relative to the viral gene, wherein the virus is an influenza virus or coronavirus.

2. The polynucleotide of claim 1, wherein the virus is an influenza A virus, influenza B virus, or influenza C virus, wherein the gene encodes an RNA polymerase or subunit thereof selected from PA, PB1, and PB2.

3. The polynucleotide of claim 2, wherein the portion of the viral gene comprises an internal deletion relative to the viral gene.

4. The polynucleotide of claim 2, wherein the nucleotide sequence comprises about 150-600 nucleotides from the 5′ end of the gene, about 150-600 nucleotides from the 3′ end of the gene, or a combination thereof, preferably wherein the nucleotide sequence comprises about 450 nucleotides from the 5′ end of the gene and about 450 nucleotides from the 3′ end of the gene.

5. The polynucleotide of claim 2, wherein at least one of the one or more defective interfering genes comprises the nucleotide sequence of any one of SEQ ID NOs:5-10, 13-15, or 17, or a nucleotide sequence having 75% or more sequence identity to any one of SEQ ID NOs: 5-10, 13-15, or 17, preferably wherein the polynucleotide collectively comprises the nucleotide sequence of SEQ ID NO:12, SEQ ID NO:13, and SEQ ID NO:14.

6. The polynucleotide of claim 1, wherein the virus is a coronavirus, preferably a β-coronavirus, more preferably SARS-CoV-2, wherein the gene encodes a structural, non-structural, accessory protein, or a fragment thereof selected from ORF1a, ORF1b, S, M, ORF3a, ORF6, ORF7a, ORFS, and ORF10.

7. The polynucleotide of claim 6, wherein the nucleotide sequence comprises about 600-1200 nucleotides from the 5′ end of the coronavirus genome, about 600-1200 nucleotides from the 3′ end of the coronavirus genome, about 600-1200 nucleotides from the gene encoding ORF1b, or a combination thereof.

8. The polynucleotide of claim 6, wherein at least one of the one or more defective interfering genes comprises the nucleotide sequence of SEQ ID NO:5 or SEQ ID NO:6, or a nucleotide sequence having 75% or more sequence identity to SEQ ID NO:5 or SEQ ID NO:6.

9. The polynucleotide of claim 1, wherein the deletion comprises about 400-2000 nucleotides or about 3,000-27,000 nucleotides, optionally wherein the nucleotides are contiguous or non-contiguous.

10. A vector comprising the polynucleotide of claim 1, wherein the vector comprises one or more promoters and/or polyadenylation signals operably linked to the one or more defective interfering genes, wherein the vector is an expression vector, preferably a plasmid.

11. A method of producing one or more defective interfering genes, the method comprising introducing the vector of claim 10 to a host cell and incubating the host cell under conditions sufficient for expression of the polynucleotide, thereby producing the one or more defective interfering genes.

12. A method of reducing replication of an influenza virus or coronavirus in a cell, the method comprising introducing the vector of claim 10 to the cell under conditions suitable for the cell to produce defective viruses comprising one or more RNAs transcribed from the polynucleotide, thereby reducing replication of the virus.

13. A composition comprising the polynucleotide of claim 1, or the vector of claim 10, wherein the composition further comprises a peptide selected from TAT-P1 (YGRKKRRQRRRCWGPCPTAFRQIGNCGRFRVRCCRIR; SEQ ID NO:3), TAT2-P1 (RKKRRQRRRCWGPCPTAFRQIGNCGRFRVRCCRIR; SEQ ID NO:4), LAH4 (KKALLAHALHLLALLALHLAHALKKA-NH2; SEQ ID NO:83), or a combination thereof.

14. The composition of claim 13, wherein the peptide:polynucleotide weight ratio is in the range of about 2:1 to 4:1, preferably, 4:1.

15. The composition of claim 13, wherein the peptide complexes with the polynucleotide to form a plurality of nanoparticles, wherein the nanoparticles have an average diameter of less than 200 nm, preferably less than 150 nm, more preferably about at least 135 nm.

16. The composition of claim 13 further comprising one or more additional polynucleotides of claim 1 or one or more additional vectors of claim 10.

17. The composition of claim 16, wherein the composition comprises three of the vectors, wherein the first vector comprises the nucleotide sequence of SEQ ID NO:12, wherein the second vector comprises the nucleotide sequence of SEQ ID NO:13, and wherein the third vector comprises the nucleotide sequence of SEQ ID NO:14.

18. A pharmaceutical composition comprising the composition of claim 13 and a pharmaceutically acceptable carrier or excipient.

19. A method of preventing or treating a viral infection or a disease associated with a viral infection in a subject, comprising administering to the subject, an effective amount of the pharmaceutical composition of claim 18, wherein the subject has been exposed to, is infected with, or is at risk of infection with an influenza virus or a coronavirus.

20. The method of claim 19, wherein the subject is immunocompromised.

21. The method of claim 19, wherein the influenza virus is influenza A virus or influenza B virus, or wherein the coronavirus is SARS-CoV-2.

22. The method of claim 21, wherein the influenza A virus is selected from H1N1, H2N2, H3N2, H3N8, H5N1, and H7N9.

23. The method of claim 21, wherein the SARS-CoV-2 is selected from SARS-CoV-2 HKU-001a, SARS-CoV-2 B.1.1.7 (Alpha variant), SARS-CoV-2 B.1.351 (Beta variant), SARS-CoV-2 B.1.617.1 (Kappa variant), SARS-CoV-2 B.1.617.2 (Delta variant), and SARS-CoV-2 B.1.617.3.

24. The method of claim 19, wherein the composition is administered via oral, intranasal, or intratracheal administration.

25. The method of claim 19, wherein the subject is human.


SEQUENCE LISTING (84 sequences)

SEQ ID NO: 1 - protein, 9 aa, synthetic construct
RKKRRQRRR

SEQ ID NO: 2 - protein, 11 aa, synthetic construct
YGRKKRRQRRR

SEQ ID NO: 3 - protein, 37 aa, synthetic construct
YGRKKRRQRRRCWGPCPTAFRQIGNCGRFRVRCCRIR

SEQ ID NO: 4 - protein, 35 aa, synthetic construct
RKKRRQRRRCWGPCPTAFRQIGNCGRFRVRCCRIR

SEQ ID NO: 5 - DNA, 2102 bp, synthetic construct
  source 1..2102: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcataacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatccgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaa

SEQ ID NO: 6 - DNA, 3600 bp, synthetic construct
  source 1..3600: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaa

SEQ ID NO: 7 - DNA, 311 bp, synthetic construct
  source 1..311: mol_type = other DNA
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaaggagagaaggctaatgtgctaattgggaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 8 - DNA, 315 bp, synthetic construct
  source 1..315: mol_type = other DNA
agcgaaagcaggcaaaccatttgaatggatgtcaatccgactttacttttcttaaaagtgccagcacaaaatgctataagcacaactttcccttatactggagaccctccttacagccatgggacaggaacaggatacaccatggatactgtcattccagagcccgaattgatgcacgaattgatttcgaatctggaaggataaagaaagaggagttcactgagatcatgaagatctgttccaccattgaagagctcagacggcaaaaatagtgaatttagcttgtccttcatgaaaaaatgccttgtttctact

SEQ ID NO: 9 - DNA, 311 bp, synthetic construct
  source 1..311: mol_type = other DNA
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 10 - DNA, 450 bp, synthetic construct
  source 1..450: mol_type = other DNA
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 11 - DNA, 450 bp, synthetic construct
  source 1..450: mol_type = other DNA
agcgaaagcaggcaaaccatttgaatggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacaattcttccccagcagttcatacagaagaccagtcgggatatccagtatggtggaggctatggtttccagagcccgaattgatgcacgaattgatttcgaatctggaaggataaagaaagaggagttcactgagatcatgaagatctgttccaccattgaagagctcagacggcaaaaatagtgaatttagcttgtccttcatgaaaaaatgccttgtttctact

SEQ ID NO: 12 - DNA, 449 bp, synthetic construct
  source 1..449: mol_type = other DNA
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatggggcaaagaagacaggagatatggaccagcattaagcataaatgaactgagcaaccttgcgaaaggagagaaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 13 - DNA, 701 bp, synthetic construct
  source 1..701: mol_type = other DNA
agcaaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgagagagtcccccaaaggagtggaggaaggttccattgggaaggtctgcagaactttattggcaaagtcggtattcaacagcttgtatgcatctccacaactggaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 14 - DNA, 596 bp, synthetic construct
  source 1..596: mol_type = other DNA
agcaaaagcaggcaaaccatttgaatggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacccgattgatgggccactgccagaagacaatgaaccaagtggttatgcccaaacagatcaaaagaaatcgatccatcttgaatacaagccaaagaggaatacttgaagatgaacaaatgtaccaaaagtgctgcaacttatttgaaaaattcttccccagcagttcatacagaagaccagtcgggatatccagtatggtggaggctatggtttccagagcccgaattgatgcacgaattgatttcgaatctggaaggataaagaaagaggagttcactgagatcatgaagatctgttccaccattgaagagctcagacggcaaaaatagtgaatttagcttgtccttcatgaaaaaatgccttgtttctact

SEQ ID NO: 15 - DNA, 596 bp, synthetic construct
  source 1..596: mol_type = other DNA
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctaactgaagacccagatgaaggcacagctggagttgagtccgcagttctgagaggattcctcattctgggcaaagaagacaggagatatggaccagcattaagcataaatgaactgagcaaccttgcgaaaggagagaaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 16 - DNA, 921 bp, synthetic construct
  source 1..921: mol_type = other DNA
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacacctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactggaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 17 - DNA, 913 bp, synthetic construct
  source 1..913: mol_type = other DNA
agcgaaagcaggcaaaccatttgaatggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacccgattgatgggccactgccagaagacaatgaaccaagtggttatgcccaaacagattgtgtattggaagcaatggccttccttgaggaatcccatcctggtatctttgagacctcgtgtcttgaaacgatggaggttgttcagcaaacacgagtggacaagctgacacaaggccgacagacctatgactggactctaaataggaaccagcctgctgcaacagcggattaccaggggcgtttatgcaacccactgaacccatttgtcaaccataaagacattgaatcagtgaacaatgcagtgataatgccagcacatggtccagccaaaaacatggagtatgatgctgttgcaacaacacactcctggatccccaaaagaaatcgatccatcttgaatacaagccaaagaggaatacttgaagatgaacaaatgtaccaaaagtgctgcaacttatttgaaaaattcttccccagcagttcatacagaagaccagtcgggatatccagtatggtggaggctatggtttccagagcccgaattgatgcacgaattgatttcgaatctggaaggataaagaaagaggagttcactgagatcatgaagatctgttccaccattgaagagctcagacggcaaaaatagtgaatttagcttgtccttcatgaaaaaatgccttgtttctact

SEQ ID NO: 18 - DNA, 915 bp, synthetic construct
  source 1..915: mol_type = other DNA
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaacctttggccctgtccattttagaaaccaagtcaaaatacacttcttcccttcgcagccgctccaccaaagcaaagtggaatgcagttctcctcattgactataaatgtgaggggatcaggaatgagaatacttgtaaggggcaattctccaatattcaactacaacaagaccactaaaagactcacagttctcggaaaggatgctggccctttaactgaagacccagatgaaggcacagctggagttgagtccgcagttctgagaggattcctcattctgggcaaagaagacaggagatatggaccagcattaagcataaatgaactgagcaaccttgcgaaaggagagaaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 19 - DNA, 1208 bp, synthetic construct
  source 1..1208: mol_type = other DNA
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtcaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactggaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 20 - DNA, 22 bp, synthetic construct
  source 1..22: mol_type = other DNA
atctgagaagacacacatccac

SEQ ID NO: 21 - DNA, 22 bp, synthetic construct
  source 1..22: mol_type = other DNA
ggactctccaacgggccatgtt

SEQ ID NO: 22 - DNA, 23 bp, synthetic construct
  source 1..23: mol_type = other DNA
caccgaaactggagcaccgcaac

SEQ ID NO: 23 - DNA, 23 bp, synthetic construct
  source 1..23: mol_type = other DNA
tttgttcatcttcaagtattcct

SEQ ID NO: 24 - DNA, 19 bp, synthetic construct
  source 1..19: mol_type = other DNA
gataacggaaatgattcct

SEQ ID NO: 25 - DNA, 19 bp, synthetic construct
  source 1..19: mol_type = other DNA
tcagaactgcggactcaac

SEQ ID NO: 26 - DNA, 23 bp, synthetic construct
  source 1..23: mol_type = other DNA
cttcgtaagaacggtaataaagg

SEQ ID NO: 27 - DNA, 22 bp, synthetic construct
  source 1..22: mol_type = other DNA
caaacaattctacatcaacacc

SEQ ID NO: 28 - DNA, 25 bp, synthetic construct
  source 1..25: mol_type = other DNA
ataatcaagactattcaaccaaggg

SEQ ID NO: 29 - DNA, 23 bp, synthetic construct
  source 1..23: mol_type = other DNA
tcattagcatgatgtctacagac

SEQ ID NO: 30 - DNA, 30 bp, synthetic construct
  source 1..30: mol_type = other DNA
cctactaaattaaatgatctctgctttact

SEQ ID NO: 31 - DNA, 21 bp, synthetic construct
  source 1..21: mol_type = other DNA
caagctataacgcagcctgta

SEQ ID NO: 32 - DNA, 28 bp, synthetic construct
  source 1..28: mol_type = other DNA
tattggtctcagggagcgaaagcaggtc

SEQ ID NO: 33 - DNA, 21 bp, synthetic construct
  source 1..21: mol_type = other DNA
ggttcttctcctgtcttcctg

SEQ ID NO: 34 - DNA, 41 bp, synthetic construct
  source 1..41: mol_type = other DNA
gaagtacacatcaggaagacaggagaagaaccggagagaac

SEQ ID NO: 35 - DNA, 34 bp, synthetic construct
  source 1..34: mol_type = other DNA
atatggtctcgtattagtagaaacaaggtcgttt

SEQ ID NO: 36 - DNA, 28 bp, synthetic construct
  source 1..28: mol_type = other DNA
tattcgtctcagggagcaaaagcaggca

SEQ ID NO: 37 - DNA, 21 bp, synthetic construct
  source 1..21: mol_type = other DNA
tgacagtatccatggtgtatc

SEQ ID NO: 38 - DNA, 42 bp, synthetic construct
  source 1..42: mol_type = other DNA
gggacaggaacaggatacaccatggatactgtcattccagag

SEQ ID NO: 39 - DNA, 33 bp, synthetic construct
  source 1..33: mol_type = other DNA
atatcgtctcgtattagtagaaacaaggcattt

SEQ ID NO: 40 - DNA, 29 bp, synthetic construct
  source 1..29: mol_type = other DNA
tattcgtctcagggagcaaaagcaggtac

SEQ ID NO: 41 - DNA, 22 bp, synthetic construct
  source 1..22: mol_type = other DNA
acttccaagtgagtgcatattg

SEQ ID NO: 42 - DNA, 43 bp, synthetic construct
  source 1..43: mol_type = other DNA
acaaatttgcagcaatatgcactcacttggaagtggctatatg

SEQ ID NO: 43 - DNA, 33 bp, synthetic construct
  source 1..33: mol_type = other DNA
atatcgtctcgtattagtagaaacaaggtactt

SEQ ID NO: 44 - DNA, 28 bp, synthetic construct
  source 1..28: mol_type = other DNA
tattggtctcagggagcgaaagcaggtc

SEQ ID NO: 45 - DNA, 23 bp, synthetic construct
  source 1..23: mol_type = other DNA
catttccgttatcctcttgtctg

SEQ ID NO: 46 - DNA, 56 bp, synthetic construct
  source 1..56: mol_type = other DNA
ccaattacagcagacaagaggataacggaaatggggcaaagaagacaggagatatg

SEQ ID NO: 47 - DNA, 34 bp, synthetic construct; identical to SEQ ID NO: 35
  source 1..34: mol_type = other DNA

SEQ ID NO: 48 - DNA, 28 bp, synthetic construct
  source 1..28: mol_type = other DNA
tattcgtctcagggagcaaaagcaggca

SEQ ID NO: 49 - DNA, 25 bp, synthetic construct
  source 1..25: mol_type = other DNA
gttgagttgcggtgctccagtttcg

SEQ ID NO: 50 - DNA, 58 bp, synthetic construct
  source 1..58: mol_type = other DNA
caacaaacaccgaaactggagcaccgcaactcaacaattcttccccagcagttcatac

SEQ ID NO: 51 - DNA, 33 bp, synthetic construct; identical to SEQ ID NO: 39
  source 1..33: mol_type = other DNA

SEQ ID NO: 52 - DNA, 29 bp, synthetic construct
  source 1..29: mol_type = other DNA
tattcgtctcagggagcaaaagcaggtac

SEQ ID NO: 53 - DNA, 23 bp, synthetic construct
  source 1..23: mol_type = other DNA
atcgccaagttctacgactattg

SEQ ID NO: 54 - DNA, 56 bp, synthetic construct
  source 1..56: mol_type = other DNA
gcaaggcgagtcaatagtcgtagaacttggcgatcaagaaaactgcttcttatcgt

SEQ ID NO: 55 - DNA, 33 bp, synthetic construct; identical to SEQ ID NO: 43
  source 1..33: mol_type = other DNA

SEQ ID NO: 56 - DNA, 28 bp, synthetic construct
  source 1..28: mol_type = other DNA
tattggtctcagggagcgaaagcaggtc

SEQ ID NO: 57 - DNA, 22 bp, synthetic construct
  source 1..22: mol_type = other DNA
ggtgataccatcactcggtctg

SEQ ID NO: 58 - DNA, 53 bp, synthetic construct
  source 1..53: mol_type = other DNA
cgccggatcagaccgagtgatggtatcacctaactgaagacccagatgaaggc

SEQ ID NO: 59 - DNA, 28 bp, synthetic construct
  source 1..28: mol_type = other DNA
tattcgtctcagggagcaaaagcaggca

SEQ ID NO: 60 - DNA, 20 bp, synthetic construct
  source 1..20: mol_type = other DNA
atctgtttgggcataaccac

SEQ ID NO: 61 - DNA, 55 bp, synthetic construct
  source 1..55: mol_type = other DNA
acaatgaaccaagtggttatgcccaaacagatcaaaagaaatcgatccatcttga

SEQ ID NO: 62 - DNA, 33 bp, synthetic construct; identical to SEQ ID NO: 39
  source 1..33: mol_type = other DNA

SEQ ID NO: 63 - DNA, 29 bp, synthetic construct
  source 1..29: mol_type = other DNA
tattcgtctcagggagcaaaagcaggtac

SEQ ID NO: 64 - DNA, 25 bp, synthetic construct
  source 1..25: mol_type = other DNA
tcatacaaatctggtagaaactttg

SEQ ID NO: 65 - DNA, 53 bp, synthetic construct
  source 1..53: mol_type = other DNA
agaaaccaaagtttctaccagatttgtatgagagagtcccccaaaggagtgga

SEQ ID NO: 66 - DNA, 33 bp, synthetic construct; identical to SEQ ID NO: 43
  source 1..33: mol_type = other DNA

SEQ ID NO: 67 - DNA, 28 bp, synthetic construct
  source 1..28: mol_type = other DNA
tattggtctcagggagcgaaagcaggtc

SEQ ID NO: 68 - DNA, 21 bp, synthetic construct
  source 1..21: mol_type = other DNA
gtattttgacttggtttctaa

SEQ ID NO: 69 - DNA, 54 bp, synthetic construct
  source 1..54: mol_type = other DNA
gccctgtccattttagaaaccaagtcaaaatacacttcttcccttcgcagccgc

SEQ ID NO: 70 - DNA, 34 bp, synthetic construct; identical to SEQ ID NO: 35
  source 1..34: mol_type = other DNA

SEQ ID NO: 71 - DNA, 28 bp, synthetic construct
  source 1..28: mol_type = other DNA
tattcgtctcagggagcaaaagcaggca

SEQ ID NO: 72 - DNA, 21 bp, synthetic construct
  source 1..21: mol_type = other DNA
gctgttgcagcaggctggttc

SEQ ID NO: 73 - DNA, 55 bp, synthetic construct
  source 1..55: mol_type = other DNA
ggactctaaataggaaccagcctgctgcaacagcggattaccaggggcgtttatg

SEQ ID NO: 74 - DNA, 33 bp, synthetic construct; identical to SEQ ID NO: 39
  source 1..33: mol_type = other DNA

SEQ ID NO: 75 - DNA, 29 bp, synthetic construct
  source 1..29: mol_type = other DNA
tattcgtctcagggagcaaaagcaggtac

SEQ ID NO: 76 - DNA, 21 bp, synthetic construct
  source 1..21: mol_type = other DNA
tgtggatgtgtgtcttctcag

SEQ ID NO: 77 - DNA, 55 bp, synthetic construct
  source 1..55: mol_type = other DNA
caataaaattaaatctgagaagacacacatccacacctccttcagtcacttcaac

SEQ ID NO: 78 - DNA, 33 bp, synthetic construct; identical to SEQ ID NO: 43
  source 1..33: mol_type = other DNA

SEQ ID NO: 79 - DNA, 29 bp, synthetic construct
  source 1..29: mol_type = other DNA
tattcgtctcagggagcaaaagcaggtac

SEQ ID NO: 80 - DNA, 23 bp, synthetic construct
  source 1..23: mol_type = other DNA
gactgacgaaaggaatcccagag

SEQ ID NO: 81 - DNA, 55 bp, synthetic construct
  source 1..55: mol_type = other DNA
ctagcagaggcctctgggattcctttcgtcagtcaatgggagaagtactgtgttc

SEQ ID NO: 82 - DNA, 33 bp, synthetic construct; identical to SEQ ID NO: 43
  source 1..33: mol_type = other DNA

SEQ ID NO: 83 - protein, 26 aa, synthetic construct
KKALLAHALHLLALLALHLAHALKKA

SEQ ID NO: 84 - DNA, 34 bp, synthetic construct; identical to SEQ ID NO: 35
  source 1..34: mol_type = other DNA